(19) United States
(12) Patent Application Publication
(10) Pub. No.: US 2020/0246308 A1
(43) Pub. Date: Aug. 6, 2020

(54) SYNERGISTIC COMBINATIONS OF UROLITHINS A AND B FOR IMPROVING COGNITIVE CAPACITY OR COGNITIVE FUNCTION

(71) Applicant: Natreon, Inc., New Brunswick, NJ (US)

(72) Inventors: Sanyasi R. Kalidindi, Monroe, NJ (US); Sairam Krishnamurthy, Varanasi (IN)

(73) Assignee: Natreon, Inc., New Brunswick, NJ (US)

(21) Appl. No.: 16/856,624

(22) Filed: Apr. 23, 2020

Related U.S. Application Data
(62) Division of application No. 16/159,343, filed on Oct. 12, 2018.

(30) Foreign Application Priority Data
Feb. 19, 2018 (IN) 201841006173

(51) Int. Cl. A61K 31/366; A61P 25/28

(52) U.S. Cl. CPC A61K 31/366; A61P 25/28

(57) ABSTRACT

A synergistic combination of Urolithin A (3,8-dihydroxy-dibenzo-alpha-pyrone) and Urolithin B (3-hydroxy-dibenzo-alpha-pyrone) is provided in a particular effective ratio, optionally in a nutraceutical or pharmaceutical composition. The composition is for use in treating cognitive deficits, including increasing cognitive function or cognitive capacity (nootropic activity). The composition is for use in treating or preventing a dementia-related disorder in a human subject, such as anxiety or Alzheimer's disease, and for inhibition of acetylcholinesterase (AChE).

This application claims priority to Indian Provisional application 201841006173, filed on Feb. 19, 2018, which is hereby incorporated by reference herein.

### FIELD OF THE INVENTION

A synergistic combination of Urolithin A (3,8-dihydroxy-dibenzo-alpha-pyrone) and Urolithin B (3-hydroxy-dibenzo-alpha-pyrone) is provided in a particular effective ratio. The present invention relates to improvement of symptoms of Alzhiemer's disease using Urolithin A and Urolithin B in combination. This invention also relates to a method of improving cognitive capacity or cognitive function using the combination.

### BACKGROUND

Alzheimer's disease (AD) is considered to be the most common form of dementia relating to memory and cognitive decline. AD is a progressive neurodegenerative disorder in which dementia symptoms gradually worsen over a number of years.

The biochemical hallmarks of AD include the accumulation of the amyloid-beta (Aβ) peptide oligomers and soluble hyperphosphorylated tau proteins. AD is also accompanied by the loss of the cholinergic markers in vulnerable neurons and the degeneration of basal forebrain cortical cholinergic neurons in end-stage AD patients. The memory loss and cognitive impairments are strongly related to changes in the acetylcholinesterase (AChE) activity. Moreover, AChE can increase the rate of fibrillation by binding amyloid-β-associated proteins as potent amyloid-promoting factors. Thus, the cholinergic hypothesis led to the development of clinically effective therapeutics for AD. Mitochondrial dysfunction is also a factor in cognitive decline.

There are a few approved drugs for treating this disease, but there is still a dire need for effective medicines.

3,8-dihdroxy-dibenzo-α-pyrone (also known as Urolithin A) and 3-hydroxy-dibenzo-α-pyrone (also known as Urolithin B) are bioactive compounds present in shilajit, which is derived from a humic exudate from sedimentary rocks. New research shows that these molecules are also metabolites of ellagitannins, generated by the microbiome in the GI tract of the animals. If a way could be found to advantageously use one or more Urlolithins to improve cognitive function, or improve cognitive capacity or even treat cognitive deficits, it would constitute a valuable contribution to the nutritional or pharmaceutical arts.

### SUMMARY

Herein are described several studies conducted on the cognitive enhancing effects of these compounds and surprisingly found that, combination of 3,8-dihdroxy-dibenzo-α-pyrone and 3-hydroxy-dibenzo-α-pyrone, in particular ratios, have a synergistic effect on anti-acetylcholinesterase activity, which in turn translates to improved cognitive capacity (nootropic activity) and/or cognitive function.

In one embodiment, the present invention describes a composition comprising urolithin A and urolithin B, wherein the wt./wt. ratio of urolithin B to urolithin A is from about 0.2:1 to about 0.6:1. Pharmaceutical or nutraceutical compositions may be prepared by including an acceptable carrier or excipient.

In an embodiment, the pharmaceutical composition is for use in a method for treating or preventing a dementia-related disorder, such as stress-induced dementia, in a human subject.

The dementia-related disorders can include, but are not limited to, anxiety or depressive disorders, Alzheimer's disease, or other cognitive disorders due to aging. The composition has an inhibitory concentration (IC₅₀) of about 0.05 micro-g/ml to about 0.06 micro-g/ml for inhibition of acetylcholinesterase activity.

### BRIEF DESCRIPTION OF THE DRAWINGS

FIG. 1 depicts Respiratory control ratio (RCR in cortical brain mitochondria) after 21 days treatment with shilajit 50 mg/kg, urolithin B 50 mg/kg, and urolithin A 50 mg/kg. Bars represent groups mean±SEM (n=5 in each group).

FIG. 2 depicts a typical bar graph representing the different states of mitochondrial respiration in cortex part of the brain mitochondria after 21 days treatment with shilajit 50 mg/kg, urolithin B 50 mg/kg and urolithin A 50 mg/kg in young and old mice. Bars represent groups mean±SEM (n=5/group).

FIG. 3 depicts a bar graph representing the effect of scopolamine (Sc) induced changes in Curiosity behavior in trial-1. All values are Mean±SEM.

FIG. 4 depicts a bar graph representing the effect of scopolamine induced changes in Curiosity behavior in trial 2. All values are Mean±SEM.

FIG. 5 depicts a bar graph representing coping behavior to a novel environment. All values are Mean±SEM. The coping behavior in rats is shown in terms of percentage of time spent in the novel arm of the apparatus during trial 2.

FIG. 6 depicts a bar graph representing number of known arm entries (%) versus novel arm entries (%) in Y-maze to determine altered arm discrimination (spatial recognition memory). All values are Mean±SEM.

FIG. 7 depicts a bar graph representing an Acetylcholinesterase (“AChE”) enzymatic inhibition assay performed according to the standard method (Ellman, G. L., et al., “A new and rapid colorimetric determination of acetylcholinesterase activity,” Biochemical Pharmacology (1961) 7: 88-95), in which shilajit (S), urolithin B (B), and urolithin A (A) were tested vs. control and compared to Donepezil (D). Values are Mean±SEM.

FIG. 8 depicts a bar graph representing an Acetylcholinesterase (“AChE”) enzymatic inhibition assay performed as in FIG. 7.

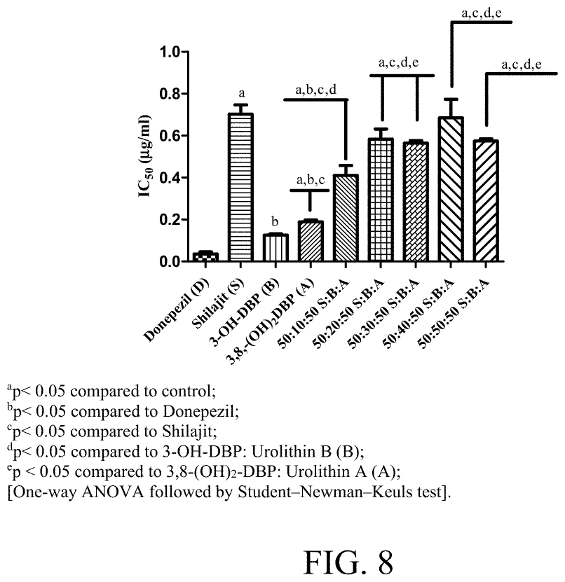

FIG. 9A depicts a bar graph illustrating inhibition of Aβ₄₀ aggregates by different stoichiometric combinations of test compounds 3(OH)-DBP (Urolithin B: B), 3,8(OH)₂-DBP (Urolithin A: A), and Shilajit denoted as compound B, A and S respectively with fixed conc. amyloid beta (10 μM) quantified by ThT fluorescence intensity, which is represented as relative fluorescence units at 485 nm for a given time point (48 h). Donepezil (D) was used as reference standard. All values are Mean±SEM.

FIG. 9B depicts a bar graph illustrating inhibition of Aβ₄₀ aggregates by different stoichiometric combinations of test compounds 3(OH)-DBP (Urolithin B: B), 3,8(OH)₂-DBP (Urolithin A: A), and Shilajit denoted as compound B, A and S respectively with fixed conc. amyloid beta (30 μM) quantified by ThT fluorescence intensity, which is represented as relative fluorescence units at 485 nm for a given time point (48 h). Donepezil (D) was used as reference standard. All values are Mean±SEM.

FIG. 10 depicts a bar graph representing a Human Recombinant Acetylcholinesterase (“AChE”) enzymatic inhibition assay performed as in FIG. 7.

FIG. 11 depicts a bar graph representing a Human Recombinant Acetylcholinesterase (“AChE”) enzymatic inhibition assay performed using the Amplex Red kit method.

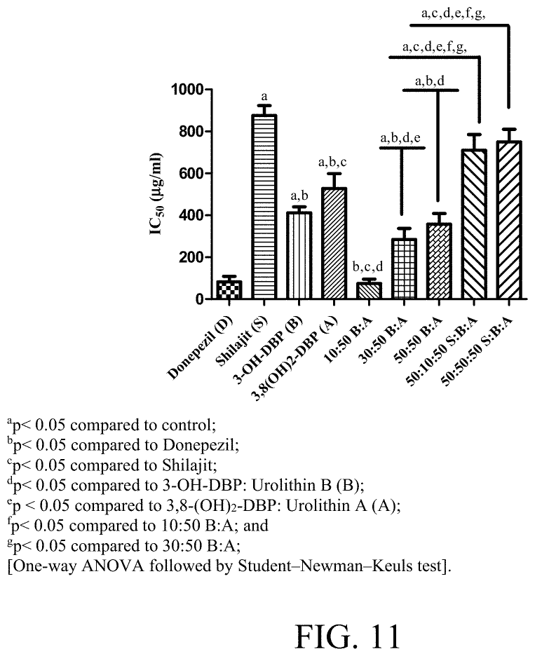

FIG. 12A depicts a bar graph illustrating inhibition of Aβ₄₀ aggregates using human recombinant AChE by different stoichiometric combinations of test compounds 3(OH)-DBP (Urolithin B: B), 3,8(OH)₂-DBP (Urolithin A: A), and Shilajit denoted as compound B, A and S respectively with fixed conc. amyloid beta (10 μM) quantified by ThT fluorescence intensity, which is represented as relative fluorescence units at 485 nm for a given time point (48 h). Donepezil (D) was used as reference standard. All values are Mean±SEM.

FIG. 12B depicts a bar graph illustrating inhibition of Aβ₄₀ aggregates using human recombinant AChE by different stoichiometric combinations of test compounds 3(OH)-DBP (Urolithin B: B), 3,8(OH)₂-DBP (Urolithin A: A), and Shilajit denoted as compound B, A and S respectively with fixed conc. amyloid beta (30 μM) quantified by ThT fluorescence intensity, which is represented as relative fluorescence units at 485 nm for a given time point (48 h). Donepezil (D) was used as reference standard. All values are Mean±SEM.

FIG. 13 depicts the experimental design of the scopolamine induced amnesic rat model testing the synergistic effects of DBPs, i.e. 3(OH)-DBP (Urolithin B: B), 3,8(OH)₂-DBP (Urolithin A: A) and their combinations, over eight (8) days.

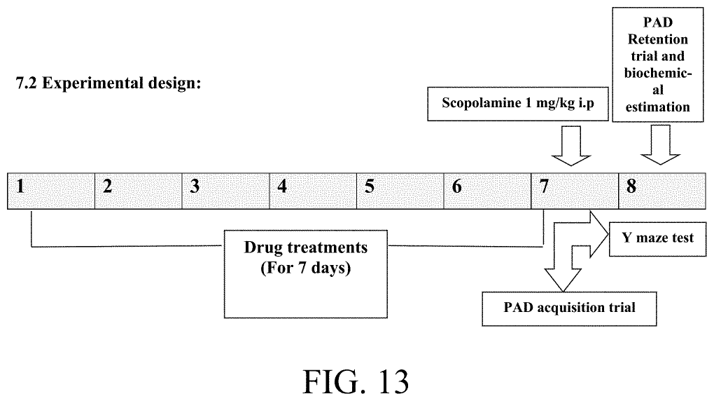

FIG. 14A depicts the effect of DBP's, i.e. 3(OH)-DBP (Urolithin B: B), 3,8(OH)₂-DBP (Urolithin A: A) and their combinations, on scopolamine (Sc)-induced changes in total arm entries during trial-1 of a scopolamine induced amnesic rat model test. All values are Mean±SEM.

FIG. 14B depicts trial 2 of the test performed as in FIG. 14A.

FIG. 14C depicts the effect of DBP's, i.e. 3(OH)-DBP (Urolithin B: B), 3,8(OH)₂-DBP (Urolithin A: A) and their combinations, on scopolamine induced changes in coping behavior to a novel environment in trial-2 of the scopolamine induced amnesic rat model test. All values are Mean±SEM.

FIG. 14D depicts the effect of scopolamine induced changes in known arm entries (%) versus novel arm entries (%) in Y-maze task of the scopolamine induced amnesic rat model test, to determine altered arm discrimination (spatial recognition memory). All values are Mean±SEM.

FIG. 15 depicts the effect of scopolamine induced memory impairment in a passive avoidance test. All values are Mean±SEM.

FIG. 16 depicts the effect of scopolamine induced changes in acetylcholinesterase (AChE) activity in hippocampal (HIP) tissue using the Amplex Red assay kit method. All values are Mean±SEM.

FIG. 17 depicts the effect of scopolamine induced changes in acetylcholine (ACh) level in HIP tissue using the Amplex Red assay kit method. All values are Mean±SEM.

### DETAILED DESCRIPTION

In one aspect, the present invention demonstrates the usefulness of shilajit and chemical constituents thereof, in treating, mitigating or preventing cognitive disorders such as dementia-related conditions, depression and/or anxiety disorders.

Shilajit is composed of rock humus, rock minerals and organic substances that have been compressed by layers of rock mixed with marine organisms and microbial metabolites. It oozes out of the rocks in the Himalayas at higher altitudes ranging from 1000-5000 meters as black mass and is regarded as a maharasa (super-vitalizer) in Ayurveda, the traditional Indian system of medicine, dating back to 3500 B.C. Shilajit contains fulvic acids as the main components along with dibenzo-α-pyrones (“DBPs”) and dibenzo-α-pyrone chromoproteins.

Two primary DBP constituent components of shilajit are 3,8-dihydroxy-dibenzo-alpha-pyrone (a.k.a. “Urolithin A”, or alternatively, “3,8-(OH)₂-DBP”) and 3-hydroxy-dibenzo-alpha-pyrone (a.k.a. “Urolithin B”, or alternatively, “3-(OH)-DBP”). Both Urolithin A and Urolithin B are available from Natreon, Inc. (New Brunswick, N.J., USA).

Fulvic acid complex, derived from shilajit, is an assembly of naturally occurring low and medium molecular weight compounds comprising oxygenated dibenzo-alpha-pyrones (DBPs), both in reduced as well as in oxidized form, as the core nucleus, and acylated DBPs and lipids as partial structural units, along with fulvic acids (“FAs”). Fulvic acid complex material derived from alluvial sources lack DBPs; instead, the core nucleus of alluvial fulvic acid is comprised of benzoic acid.

Thus, the active constituents of shilajit contain dibenzo-alpha-pyrones and related metabolites, small peptides (constituting non-protein amino acids), some lipids, and carrier molecules (fulvic acids). See, Ghosal, S., et al., “Shilajit Part 1—Chemical constituents,” J. Pharm. Sci. (1976) 65:772-3; Ghosal, S., et al., “Shilajit Part 7—Chemistry of Shilajit, an immunomodulatory ayurvedic rasayana,” Pure Appl. Chem. (IUPAC) (1990) 62:1285-8; Ghosal, S., et al., “The core structure of Shilajit humus,” Soil Biol. Biochem. (1992) 23:673-80; and U.S. Pat. Nos. 6,440,436 and 6,869,612 (and references cited therein); all hereby incorporated by reference herein.

Shilajit (e.g., PrimaVie®) finds extensive use in Ayurveda, for diverse clinical conditions. For centuries, people living in the isolated villages in Himalayas and adjoining regions have used Shilajit alone, or in combination with, other plant remedies to prevent and combat problems with diabetes (Tiwari, V. P., et al., “An interpretation of Ayurvedica findings on Shilajit,” J. Res. Indigenous Med. (1973) 8:57). Moreover being an antioxidant it will prevent damage to the pancreatic islet cell induced by the cytotoxic oxygen radicals (Bhattacharya S. K., “Shilajit attenuates streptozotocin induced diabetes mellitus and decrease in pancreatic islet superoxide dismutase activity in rats,” Phytother. Res. (1995) 9:41-4; Bhattacharya S. K., “Effects of Shilajit on biogenic free radicals,” Phytother. Res. (1995) 9:56-9; and Ghosal, S., et al., “Interaction of Shilajit with biogenic free radicals,” Indian J. Chem. (1995) 34B:596-602). It has been proposed that the derangement of glucose, fat and protein metabolism during diabetes, results into the development of hyperlipidemia. In one study, Shilajit produced significant beneficial effects in lipid profile in rats (Trivedi N. A., et al., “Effect of Shilajit on blood glucose and lipid profile in alloxan-induced diabetic rats,” Indian J. Pharmacol. (2004) 36(6):373-376).

As discussed, shilajit has been used to treat various ailments. It is also recommended as a performance enhancer. Fulvic acids (FAs) are reported to elicit many important roles in biological systems of plants, in animals as well as humans, including: (a) improvement of bioavailability of minerals and nutrients, (b) serve as electrolytes, (c) detoxification of toxic substances including heavy metals, (d) perform as antioxidants, and (e) improvement of immune function. Furthermore, dibenzo-α-pyrones have been hypothesized to participate in the electron transport inside the mitochondria, thus facilitating production of more ATP, leading to increased energy.

In one aspect, the present invention demonstrates the usefulness of 3-hydroxy-dibenzo-α-pyrone (3-OH-DBP), 3,8-dihydroxy-dibenzo-α-pyrone (3,8-(OH)₂-DBP), or combinations thereof in treating human individuals suffering from various cognitive deficits or a decline in cognitive capacity or function.

In order to evaluate the antiaging effects of 3,8-dihdroxy-dibenzo-α-pyrone (Urolithin A), 3-hydroxy-dibenzo-α-pyrone (Urolithin B), and shilajit, the following studies were carried out:

Study 1: Effect of the experimental drugs on mitochondrial bioenergetics and function in young and aged mice;

Study 2: Effect of the experimental drugs on scopolamine-induced dementia in rats;

Study 3: Effect of 3,8-dihdroxy-dibenzo-α-pyrone (Urolithin A) and 3-hydroxy-dibenzo-α-pyrone (Urolithin B), and their combinations in different ratios, on acetylcholinesterase (AChE) activity in-vitro.

### Example 1 (Study 1)

1. Objective of Study 1: To Study the Experimental Drugs on Mitochondrial Bioenergetics and Function in Young and Aged Mice.

1.1. The Experimental Design of the Study is Shown in Table 1.

TABLE 1

| Sample No. | Study Group (Dose) |
|---|---|
| 1 | Young control group (6 to 8 week) |
| 2 | Old control group (18 to20 month) |
| 3 | Young group + shilajit (50 mg/kg p.o.) |
| | (For 21 days) |
| 4 | Young group + Urolithin B (50 mg/kg p.o.) |
| | (For 21 days) |
| 5 | Young group + Urolithin A (50 mg/kg p.o.) |
| | (For 21 days) |
| 6 | Old group + shilajit (50 mg/kg p.o.) |
| | (For 21 days) |
| 7 | Old group + Urolithin B (50 mg/kg p.o.) |
| | (For 21 days) |
| 8 | Old group + Urolithin A (50 mg/kg p.o.) |
| | (For 21 days) |

2. Materials and Methods

2.1. Chemicals

Shilajit, Urolithin B, and Urolithin A were provided by Natreon, Inc. (New Brunswick, N.J., USA). Mannitol, sucrose, bovine serum albumin (BSA), are available from HiMedia Pvt Ltd. (Mumbai, India). EGTA, HEPES potassium salt, potassium phosphate monobasic anhydrous (KH₂PO₄), MgCl₂, malate, pyruvate, ADP, succinate, oligomycin, carbonyl cyanide 4-(trifluoromethoxy)phenylhydrazone (FCCP), and Rotenone were procured from Sigma-Aldrich (St. Louis, Mo., USA).

3. Evaluation of Mitochondrial Bioenergetics

3.1. Mitochondrial Isolation

Isolation of mitochondria from the cortex of mouse brain was done by standard differential centrifugation as per the method of previously described (Berman, S. B., Hastings, T. G., “Dopamine oxidation alters mitochondrial respiration and induces permeability transition in brain mitochondria.” J. Neurochem. (1999) 73: 1127-1137) with some slight modifications (Samaiya, P. K., Krishnamurthy, S., “Characterization of mitochondrial bioenergetics in neonatal anoxic model of rats.” J. Bioenerg. Biomembr. (2015) 47: 217-222). Briefly, brains were dissected and were homogenized in isolation buffer (consisting of 215 mM mannitol, 75 mM sucrose, 0.1% w/v bovine serum albumin, 20 mM HEPES buffer and 1 mM of EGTA in 100 ml of distilled water and pH adjusted to 7.2 with KOH) and first centrifuged at 1300 g for 3 min. The supernatant was stored as S1 and pellet were again centrifuged as above to collect S2. S1 and S2 then were mixed and each supernatant was then topped off with isolation buffer with EGTA and centrifuged at 14,000×g for 10 min at 4° C. to get a tighter mitochondrial pellet. Next, a washing step was performed by suspending the pellets in isolation buffer without EGTA and again centrifuged at 14,000×g for 10 min to remove EGTA from the pellets. Mitochondrial protein was estimated calorimetrically (Lowry, O. H., et al., “Protein measurement with the Folin phenol reagent.” J. Biol. Chem. (1951) 193:265-275) with a microplate reader (Biotek, USA).

3.2. Measurement of Mitochondrial Function

Mitochondrial respiration was assessed as described previously (Samaiya and Krishnamurthy, 2015) with a miniature Clark-type electrode in a sealed, thermostatically controlled chamber at 37° C. (Hansatech, Norfolk, U.K.). Briefly the mitochondria were added to the chamber and respiratory states were evaluated by suitable substrates and inhibitors. Purified mitochondrial protein was suspended in respiration buffer in a final volume of 250 μL. State II respiration was initiated by addition of Pyruvate/Malate (P/M), with basal rate of respiration. State III respiration was initiated by addition of ADP; the high level of oxygen utilization indicates that ADP is getting converted into ATP. State IV was measured by addition of oligomycin. The respiration returns to basal rate since the ATP synthase is shut down and no electrons are allowed to return to the matrix. The electron transport chain (ETC) continues only to maintain mitochondrial membrane potential due to loss of protons back into the matrix. State V was measured by addition of FCCP. This represents the maximum rate of respiration, causing uncoupling of the ETC to ATP synthesis, and allows protons to rush back into the matrix. Rotenone was then added to shut down complex I-driven respiration. State V (succinate) was determined by addition of succinate. This is the maximum rate of respiration via complex II, since FCCP is present in the system. The respiratory control ratio (RCR) was calculated by dividing the slope of the response of isolated mitochondria to state III respiration (presence of ADP) by slope of the response to state IV respiration (presence of 1 IM oligomycin and absence of ADP (Samaiya and Krishnamurthy, 2015).

4. Results and Discussion

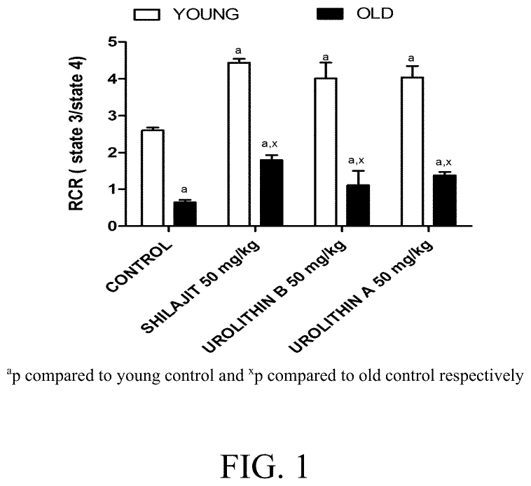

A. Effects of shilajit, urolithin B and urolithin A on mitochondrial bioenergetics (RCR) in young and old mice are shown in FIG. 1. Statistical analysis by two-way ANOVA revealed that there were significant differences in RCR within the group (row factor) [F (3, 32)=13.16, P<0.05] and between the group (column factor) [F (1, 32)=212.20, P<0.05]. Bonferroni post-hoc test showed there was significant decrease in RCR with age. Old rats had lower RCR compared to younger rats. This indicates that there is significant compromise in mitochondrial bioenergetics in old rats. Shilajit, Urolithin B and Urolithin A (50 mg/kg) significantly increased the RCR in old mice indicating that these compounds can ameliorate deranged mitochondrial bioenergetics ion old rats. Further there was a significant increase in RCR even among young rats with treatment indicating that these compounds could be used as a food supplement and also as a drug in disorders related to aging, respectively; however mitochondrial function was improved in both young and old mice during the 21 days treatment.

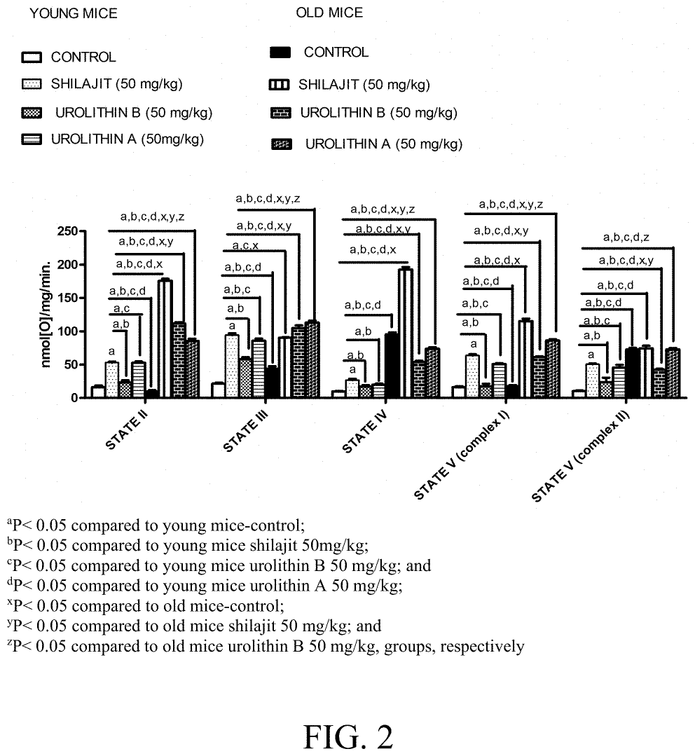

B. The effects of shilajit, urolithin B and urolithin A (50 mg/kg) on the different states of mitochondrial respiration in young and old mice are shown in FIG. 2. Two-way ANOVA revealed that there were significant differences in different states within the group (row factor) [F (4, 80)=7.5, P<0.05] and between the group (column factor) [F (4, 28)=335, P<0.05]. Bonferroni post-hoc analysis showed that shilajit, urolithin B and urolithin A (50 mg/kg) demonstrated significantly increased mitochondrial respiration in all the state in comparison to normal control mice with their respective groups during 21 days treatment.

It is more likely that aging results from the accumulation of unrepaired damage to somatic cellular components. Free radicals produced during the normal process of oxidative phosphorylation have been proposed to be a major source of cellular damage and the free radical theory of aging was first proposed over 50 years ago (Harman, D., “Ageing: a theory based on free radical and radiation chemistry,” J. Gerontol. (1956) 11:298-300). The mitochondrial theory of aging (Harman, D., “The biologic clock: the mitochondria?” J. Am. Geriatr. Soc. (1972) 20: 145-147.), was proposed following a study which reported that the majority of ROS production in the cell was from the mitochondria (Chance, B., et al., “Hydroperoxide metabolism in mammalian organs,” Physiol. Rev. (1979) 59: 527-605.). The examples described herein assessed the anti-aging effect with reference to mitochondrial function of the following drugs during 21 days of treatment schedule with the dose of 50 mg/kg in young (6 to 8 week) mice having 10-15 gm weight and old (18 to 20 month) mice having 20-25 gm weight. Subsequently the respiratory control ratio (RCR) was calculated by state III dividing with state IV respiratory rate. (Gilmer, L. K., et al., “Age-related changes in mitochondrial respiration and oxidative damage in the cerebral cortex of the Fischer 344 rat,” Mech. Ageing Dev. (2010) 131:133-143). In the 21 days of experimental schedule, it was found that shilajit, urolithin B and urolithin A in doses of 50 mg/kg, improved the mitochondrial complex enzyme activity, mitochondrial respiratory rate and delayed the aging in the young mice and reduced the rate of aging in old mice.

The state II respiration depicts the consumption of substrate Pyruvate/Malate to fuel the mitochondrial electron transport chain (ETC), and there were significant increases in respiratory rate observed in young mice treated with shilajit, urolithin B and urolithin A at doses of 50 mg/kg when compared to young control mice. The respiratory rate is also found to be increased in old mice treated with shilajit, urolithin B and urolithin A at doses of 50 mg/kg when compared to old control mice. Whereas, increase in respiratory rate is more significant in old mice treated with all the three compounds than young mice treated with the same. State III in isolated mitochondria is a state with high external (extra mitochondrial) ADP, low external ATP/ADP ratio and high (maximal in isolated mitochondria without Ca²⁺) oxygen consumption and ATP synthesis (Chance B., Williams G. R., “Respiratory enzymes in oxidative phosphorylation I. Kinetics of oxygen utilization,” Journal of Biological Chemistry. (1955) November 1; 217(1):383-94; and Chance B., Williams G. R., “The respiratory chain and oxidative phosphorylation,” Adv. Enzymol. (1956) 17: 65-134). State III respiration was initiated by addition of ADP. In this study, state III respiration were also found to be same as state II. On the other hand, State IV is defined as a state with a very high ATP/ADP ratio, very low ADP, no ATP synthesis and oxygen consumption corresponding exclusively to proton leak (Groen, A. R., et al., “Quantification of the contribution of various steps to the control of mitochondrial respiration,” J. Biol. Chem. (1982) 257: 2754-2757; Hafner, R. P., et al., “Analysis of the control of respiration rate, phosphorylation rate, proton leak rate and proton motive force in isolated mitochondria using the ‘top-down’ approach of metabolic control theory,” Eur. J. Biochem. (1990) 188: 313-319; Schild, L., Gellerich F. N., “Effect of the extramitochondrial adenine nucleotide pool size on oxidative phosphorylation in isolated rat liver mitochondria,” Eur. J. Biochem. (1988) 252: 508-512; and Wanders, R. J. A., et al., “Factors determining the relative contribution of the adenine-nucleotide translocator and the ADP-regenerating system to the control of oxidative phosphorylation in isolated rat-liver mitochondria,” Eur. J. Biochem. (1984) 142: 417-424). An increase in state IV respiration was found by using oligomycin (ATP-synthase inhibitor) in both the young and old group mice; but in old mice this increasing effect in respiratory rate is more prominent than young mice. Moreover, shilajit in doses of 50 mg/kg shows significant effect in state IV respiration as well. The increase in state IV respiration may be due to damage of the inner mitochondrial membrane which causes more protons to leak into the matrix. This results in loss of maximal proton gradient which is required to couple with ATP production. As State IV respiration denotes the extent of coupling of proton motive force with ATP production versus maintaining the basal metabolic rate, increase in oxygen utilization denotes uncoupling of the ETC to ATP production. (See, Samaiya and Krishnamurthy, 2015). Subsequent addition of a mild uncoupler FCCP (state V) detaches the ETC from oxidative phosphorylation. This determines the maximum respiration capabilities of the ETC in its attempts to restore the dissipated proton gradient (Benz, R., McLaughlin, S., “The molecular mechanism of action of the proton ionophore FCCP (carbonylcyanideptrifluoromethoxyphenylhydrazone),” Biophys. J. (1983) 41:381-398). As the mitochondria were not able to overcome this effect and maintain respiration due to loss in function of complex I. the respiratory rate at state V (complex I) was significantly found to be increased in young mice treated with shilajit, urolithin B and urolithin A. However, urolithin B had less effect in this state probably due to its action as an uncoupler for the state V (complex I). In old mice all the three drugs show an increase in respiration rate. Following the addition of rotenone, which acts as a competitive inhibitor to block complex I driven respiration, succinate was added to determine if complex II driven respiration is affected by the aging. There was significant loss of Complex-II state V respiration observed in old group mice but not in young mice. This result indicates that susceptibility of complex-II for the aged mice. RCR is the ratio of state III to state IV respiration and it is a measure of mitochondrial integrity (Gilmer L. K., et al., 2010). There was significant increase in RCR found in all three drugs at the dose of 50 gm/kg in both young and old experimental mice. From the above result it can be concluded that the experimental drugs demonstrated improved bioenergetics in both young and old mice.

5. Conclusion

On the basis of present study, Shilajit, Urolithin B and Urolithin A in the dose of 50 mg/kg body weight in mice improved the mitochondrial bioenergetics and function in both young and old mice.

### Example 2 (Study 2)

1. Objective of Study 2: To Evaluate Effect of Experimental Drugs on Scopolamine Induced Dementia in Rats.

2. Methods

2.1. Drugs. Experimental Drugs: 3-OH-DBP (Urolithin B), 3,8-(OH)₂-DBP (Urolithin A), and Shilajit were Procured from Natreon, Inc. (New Brunswick, N.J., USA).

2.2. Animal subjects. Inbred Charles Foster male albino rats (130-180 g) were obtained from the Central Animal House, Institute of Medical Sciences (IMS), Banaras Hindu University (BHU), Varanasi, Uttar Pradesh, India. These were certified to be pathogen free and were randomly distributed into different experimental groups. The rats were kept in the departmental animal house at an ambient temperature of 25±1° C. and 45-55% RH, with a 12-h light/dark cycle (lights on 7:00-19:00 h). The animals had free access to standard pellet chow and tap water ad libitum. Experiments were conducted between 9:00 and 14:00 h. The behavioral testing was done during the light phase. Animals were housed in groups and acclimatized for at least one week before using them for experiments. Animals were transported in cage covered by dark cloth to the experimental room. The experimental procedures were in compliance with National Institutes of Health Guide for Care and Use of Laboratory Animals.

2.3. Drug Treatment.

The rats were pretreated orally for seven days through an orogastric tube with experimental drugs (10, 25 and 50 mg/kg once daily) suspended in 0.3% carboxymethyl cellulose (CMC). The treatment was continued until the end of the experiment procedures (Singh, G. K., Garabadu, D., Muruganadam, A. V., Joshi, V. K., Krishnamurthy, S., “Antidepressant activity of Asparagus racemosus in animal models of depression,” Pharmacol. Biochem. Behav. (2009) 91(3):283-90). Scopolamine was dissolved in water for injection and administered on day 7 after one hour of drug treatment administration. Donepezil (5 mg/kg; i.p.)

was used as standard drug. On day 8 the behavioral experiment was performed. The animals were sacrificed and hippocampus was micro-dissected using the coordinates of the rat brain atlas. All control group animals were treated with the 0.3 ml of vehicle (0.3% CMC suspension) equal to the volume of experimental drugs (Ohj a, R., et al., “Asparagus recemosus enhances memory and protects against amnesia in rodent models,” Brain Cogn. (2010) October; 74(1): 1-9).

2.4. Experimental Method

Spatial recognition memory, general exploratory behavior, and anxiety-like behavior were assessed by the Y-maze test. Y-maze consists of three similar arms of dimension 50 cm long, 16 cm wide and 32 cm high. In the first trial, the novel arm was blocked and the animal was allowed to move for 15 min in other two arms. In the second trial, the novel arm was opened and the animal was allowed to move for 5 min in all three arms after 4 h of the first trial. The total number of entries in all arms (for 5 min of trial-1 and trial-2) is indicative of general exploration attitude (curiosity). The % entries in known and novel arms for the 5 min period of trial 2 were considered as a measure of spatial recognition memory. Coping strategy to the novel environment was estimated by the percentage of the ratio of time spent in the novel arm to time spent in all arms and in the center of the apparatus during trial 2. The decrease in the coping behavior to the novel environment was considered as an increase in anxiety-like behavior (Krishnamurthy, S., et al., “Risperidone ameliorates post-traumatic stress disorder-like symptoms in modified stress re-stress model,” Neuropharmacology (2013) 75:62-77).

3. Results and Discussion

A. Effect of 3-OH-DBP (Urolithin B) and 3,8-(OH)₂-DBP (Urolithin A) on Curiosity Behavior in Y-Maze Test.

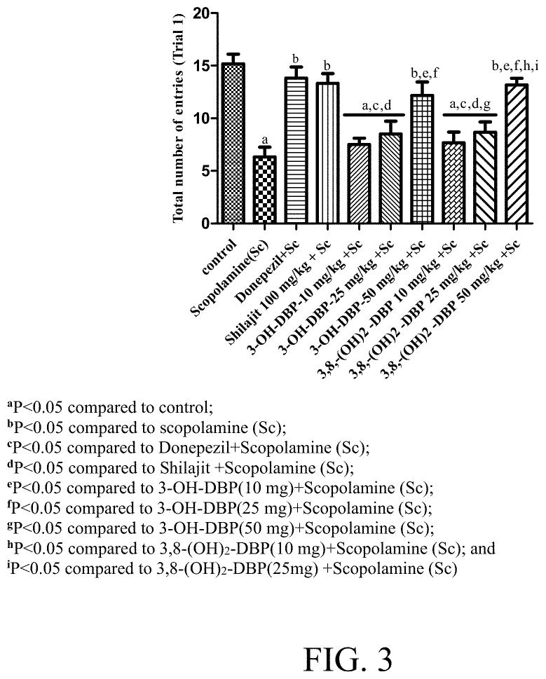

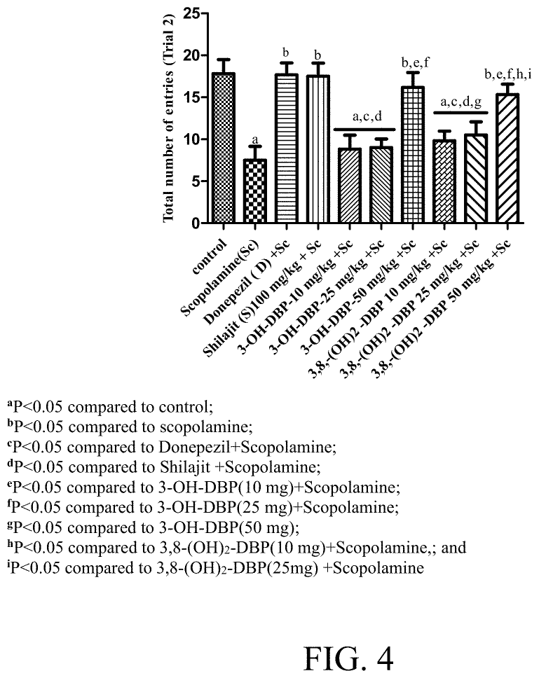

FIGS. 3 and 4 show the total number of entries in trial-1 and trial-2, i.e., curiosity behavior in rats subjected to the Y-maze test. Statistical analysis showed that there that there were significant differences among groups [F (9, 59)=10.63p<0.05], [F (9, 59)=7.984, p<0.05] in trial-1 and trial-2 respectively. One-way ANOVA followed by Student-Newman-Keuls test. Student Newman-Keuls test suggests that scopolamine administration caused a significant decrease in curiosity behavior as compare to control group rat. However, 3-OH-DBP at the 50 mg dose and 3,8-(OH)₂-DBP at the 50 mg dose attenuated the scopolamine induced decrease in curiosity behavior in trial-1 and trial-2.

B. Effect of 3-OH-DBP (Urolithin B) and 3,8-(OH)₂-DBP (Urolithin A) on Coping Behavior to Novel Environment in Y-Maze Test.

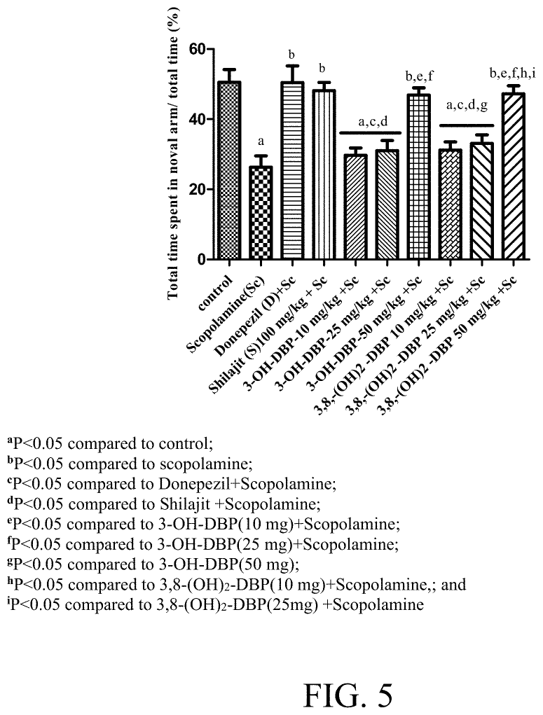

FIG. 5 shows the coping behavior in rats in terms of percentage of time spent in the novel arm of the apparatus during trial-2. Statistical analysis revealed that there were significant differences among groups [F (9, 59)=11.53, p<0.05]. One-way ANOVA followed by Student-Newman-Keuls test. Student Newman-Keuls test suggests that there was a significant loss of coping behavior in scopolamine treated group animals compared to control group rats. 3-OH-DBP at the 50 mg dose and 3,8-(OH)₂-DBP at the 50 mg dose attenuated the scopolamine-induced decrease in coping strategy to a novel environment.

C. Effect of 3-OH-DBP (Urolithin B) and 3,8-(OH)₂-DBP (Urolithin A) on Alterations in Arms Discrimination Behavior in Y-Maze Test.

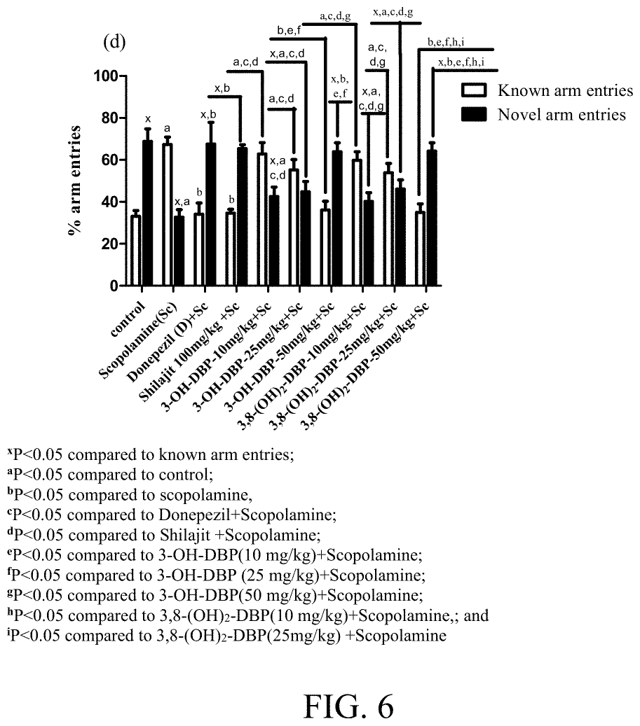

The effect on spatial memory impairment after 7 days of scopolamine induction in the Y-maze test is depicted in FIG. 6. Statistical analysis by Two-way ANOVA revealed that there were significant differences for % known and novel arm entries among groups [F (9, 100)=0.072, p<0.05]. Two-way ANOVA followed by Bonferroni-post-hoc-test. Post hoc analysis revealed that there were a significant increase and a decrease in the % known and novel arm entries, respectively, in scopolamine-treated rats. 3-OH-DBP at the 50 mg dose and 3,8-(OH) 2-DBP at the 50 mg treatment dose each dose dependently reversed the % known and novel arm entries in the Y-maze test paradigm as compare to scopolamine treated rats.

4. Summary and Conclusion

Scopalamine control reduced the total arm entries in the Y-Maze indicating aberrant changes in the exploratory behavior of the animals thus providing a mammalian model of cognitive deficiency. Exploratory behavior is supposed to be survival instinct of the rodents in novel environment. The animal tries to gather information about their surrounding for potential threats and material gain. The balance between the threat and the gain influences the exploratory behavior. This motivational behavior may be altered by internal physiological changes or by external factors such as stress. In the present study, scopalamine altered this behavior by down regulating the cholinergic system. Donepezil, an anticholinesterase drug, reversed this activity by probably increasing the synaptic availability of acetylcholine. Without being bound by theory in the present disclosure, it is believed that by the same mechanism both the DBP's (namely, urolithins A and B) and shilajit attenuated the altered scopolamine-induced exploratory behavior. DBP's treatment dose dependably reversed the known arm and novel arm entries in the Y-maze test indicating attenuation of scopolamine-induced spatial memory impairment. Further, scopolamine treatment caused significant loss of coping behavior in terms of time spends in the novel environment indicating anxiety-like behavior. DBP's attenuated the scopolamine-induced decrease in coping strategy to a novel environment. Thus, DBP's clearly demonstrated anxiolytic activity in the Y-maze paradigm (Krishnamurthy, S., Garabadu, D., Joy, K. P. “Risperidone ameliorates post-traumatic stress disorder-like symptoms in modified stress re-stress model,” Neuropharmacology (2013) 75:62-77; and Tripathi, A., Paliwal, P., Krishnamurthy, S. “Piracetam Attenuates LPS-Induced Neuroinflammation and Cognitive Impairment in Rats,” Cellular and Molecular Neurobiology, (2017) 1-14). Therefore, DBP's improves cognitive deficits and exhibits anxiolytic activity in scopolamine-induced dementia model. It is interesting to note that the effects of DBP's was at a 50 mg/kg dose and shilajit at a 100 mg/kg dose. Therefore, DBP's seem to be twice as potent as shilajit in the above observed pharmacological effects.

### Example 3 (Study 3)

1. Objectives of Study 3: To assess the Effect of Urolithin B (B) and Urolithin A (A), and their combinations in different ratios on acetylcholinesterase (AChE) activity in-vitro (Ellman, G. L., et al., “A new and rapid colorimetric determination of acetylcholinesterase activity,” Biochemical Pharmacology (1961) 7: 88-95. To determine synergistic effects of DBP's on AChE activity.

Shilajit, Urolithin B (B), and Urolithin A (A) were provided by Natreon, Inc. (New Brunswick, N.J., USA). AchE from Electrophorus electricus (electric eel) CAS: 9000-81-1 was procured from Sigma Aldrich (St. Louis, Mo., USA). 5,5′-dithiobis (2-nitrobenzoic acid) (DTNB-Ellman's reagent) and acetylcholine iodide, (AChI) were procured from local suppliers (HiMedia, Pvt. Ltd., New Delhi, India). All other chemicals and reagents were available commercially from local suppliers (Merck Pvt. Ltd., New Delhi and HiMedia Laboratories Pvt Ltd., New Delhi, India) and were of analytical grade.

The procedure described by Ellman et al. was used after some modification for AChE inhibition assay. Acetylthiocholine iodide (ATCI), 5,5′-dithiobis (2-nitrobenzoic acid) (DTNB-Ellman's reagent) were purchased from Himedia. Donepezil was used as positive control. Tris-HCl buffer (pH 8) was used to perform the in-vitro assay. Percentage inhibition was determined at 50 mg/ml to decide the concentration range for IC₅₀ assay. Five different combination concentration ranges for test compound B (3-OH-DBP): test compound A (3,8 (OH)₂ DBP) were used and the ratios were: Concentration of test compound A 50 mg was varied with test compound B (10, 20, 30, 40, 50 mg) were used to determine the IC₅₀. 50 μL of AChE (0.22 U mL⁻¹) and 10 μL of test or standard compounds were incubated in 96 well plate for 30 min. at room temperature (See, Table 3).

Further, substrate i.e. ATCI (15 mM, 30 μL) was added and it was incubated for additional 30 min. Finally 160 μL (1.5 mM) of DTNB was added and the absorbance was recorded at 415 nm using Epoch microplate reader (BioTek). The IC₅₀ value was calculated using absorbance obtained from the test and standard compounds. The assay was performed in triplicate and in three independent runs.

Percentage inhibition=(Control−Test)/Control×100 Equation (1)

1.1. The Experimental Design of the Study is Shown in Table 2.

TABLE 2

| Sample No. | Group | IC₅₀ (μg/ml) |
|---|---|---|
| 1 | Control | 0.0000 ± 0.0000 |
| 2 | Donepezil | 0.0366 ± 0.0094 |
| 3 | Shilajit | 0.6030 ± 0.0441 |
| 4 | Urolithin B (B) | 0.1430 ± 0.0057 |
| 5 | Urolithin A (A) | 0.1793 ± 0.0092 |
| 6 | 10:50 B:A | 0.0459 ± 0.0079 |
| 7 | 20:50 B:A | 0.0546 ± 0.0080 |
| 8 | 30:50 B:A | 0.0575 ± 0.0089 |
| 9 | 40:50 B:A | 0.1036 ± 0.0119 |
| 10 | 50:50 B:A | 0.1260 ± 0.0188 |

2. Results

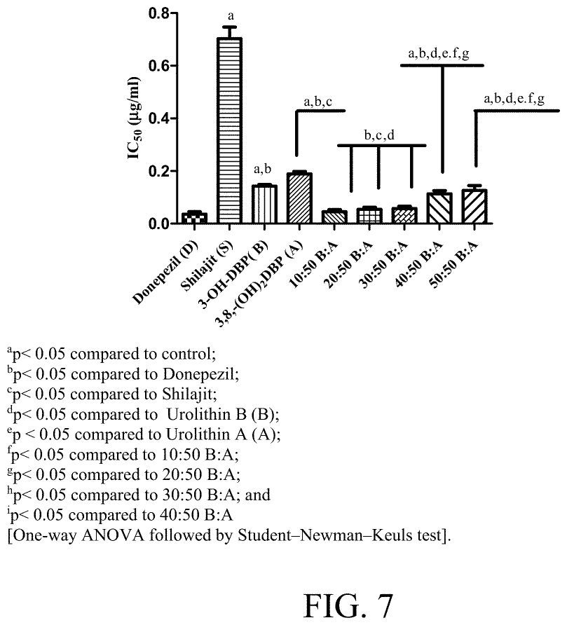

As shown in Table 2 and depicted in FIG. 7, combination doses 10:50, 20:50 and 30:50 show approximately 4 times more effective IC₅₀ value than 3,8 (OH)₂ DBP and 3 times more effective than 3-OH-DBP. Donepezil was used as reference standard, whose IC₅₀ value approximately was similar to that 10:50, 20:50 and 30:50. Shilajit has 20 times less effective IC₅₀ value than 10:50 and 8 times less than 3-OH-DBP and 5 times less than 3,8 (OH)₂ DBP. The IC₅₀ value of Shilajit was 25 times less effective than Donepezil. These data revealed that 3-OH-DBP and 3,8 (OH)₂DBP show synergistic effects at 10:50, 20:50 and 30:50 concentrations.

Furthermore, Table 2 demonstrates that a particular ratio of Urolithin B to Urolithin A unexpectedly has about a three-fold to four-fold increase in AChE inhibitory activity, over each component, alone. This synergistic combination of Urolithin B with Urolithin A has almost similar activity compared to Donepezil, a known prescription drug.

Table 3 shows certain advantageous and effective combinations of Urolithin B with Urolithin A as described herein, reflecting the combinations and ratios of Table 2. Additional effective concentration ranges are contemplated.

TABLE 3

| Urolithin B (B) (in mg/ml) | Urolithin A (A) (in mg/ml) |
|---|---|
| 10 | 50 |
| 20 | 50 |
| 30 | 50 |
| 40 | 50 |
| 50 | 50 |

In an embodiment the combination of Urolithin B to Urolithin A can be prepared as a pharmaceutical or neutraceutical formulation. Exemplary wt./wt. ratios of Urolithin B to Urolithin A can range from about 0.2:1 to about 1:1. In a preferred embodiment, the wt./wt. ratios of Urolithin B to Urolithin A can range from about 0.2:1 to about 0.6:1.

In accordance with the examples as described herein, a daily dose of the aforementioned synergistic combination(s) of Urolithin B and Urolithin A can range from about 1.5 mg/kg to about 8.0 mg/kg in a human subject. In another embodiment, the daily dose can range from about 1.5 mg/kg to about 10.0 mg/kg in a human subject.

In one embodiment, a daily dose of the aforementioned synergistic combination(s) of Urolithin B and Urolithin A can range from about 100 mg to about 1000 mg in a human subject. In a preferred embodiment, a daily dose of the aforementioned synergistic combination(s) of Urolithin B and Urolithin A can range from about 100 mg to about 500 mg in a human subject.

It is further expected that the synergism observed in Study 3 will be exhibited in a similar manner in Studies 1 and 2, when carried out in a comparable dose range in a mammalian subject. It is further expected that the Urolithin B/Urolithin A synergy will be observed in a method for treating or preventing a dementia-related disorder in a human subject, or in a method for treating or preventing an anxiety disorder in a human subject.

### Example 4

In an embodiment, the objective of this study is to assess the in-vitro synergistic effect of Shilajit with DBP's in various combinations on Acetylcholinesterase activity by using the Ellman (1961) method. Chemical reagents and starting materials were employed as in Example 3, as was the AChE inhibition assay.

Five different combinations of test compounds urolithin A, urolithin B, and shilajit were made as follows. The concentrations of test compound A (50 mg) and shilajit (50 mg) were combined with varying concentrations of test compound B to determine IC₅₀ values, as shown in Table 4. 50 μL of AChE (0.22 U mL⁻¹) and 10 μL of test or standard compounds were incubated in 96 well plate for 30 min. at room temperature.

TABLE 4

| Shilajit | Test compound Urolithin B | Test compound Urolithin A |
|---|---|---|
| (in mg/ml) | (in mg/ml) | (in mg/ml) |
| 50 | 10 | 50 |
| 50 | 20 | 50 |
| 50 | 30 | 50 |
| 50 | 40 | 50 |
| 50 | 50 | 50 |

Further, substrate i.e. ATCI (15 mM, 30 μL) was added and it was incubated for additional 30 min. Finally 160 μL (1.5 mM) of DTNB was added and the absorbance was recorded at 415 nm using Epoch microplate reader (BioTek). The IC₅₀ value was calculated using absorbance obtained from the test and standard compounds. The assay was performed in triplicate (see Equation 1). Results are shown in Table 5 below.

TABLE 5

| | Concentration ratio | |
|---|---|---|
| Sample No. | Urolithin B (B):Urolithin A (A) | IC₅₀ (μg/ml) |
| 1 | Control | 0.0000 ± 0.0000 |
| 2 | Donepezil | 0.0366 ± 0.0094 |
| 3 | 3-OH-DBP (B) | 0.1430 ± 0.0057 |
| 4 | 3-8,-(OH)2-DBP (A) | 0.1793 ± 0.0092 |
| 5 | Shilajit (S) | 0.6030 ± 0.0441 |
| 6 | (S:B:A) 50:10:50 | 0.4104 ± 0.0471 |
| 7 | (S:B:A) 50:20:50 | 0.5845 ± 0.0474 |
| 8 | (S:B:A) 50:30:50 | 0.5649 ± 0.0112 |
| 9 | (S:B:A) 50:40:50 | 0.6857 ± 0.0879 |
| 10 | (S:B:A) 50:50:50 | 0.5742 ± 0.0108 |

In conclusion, the combination dose shilajit with DBP's 50:10:50 shows approximately 1.5 times more effective IC₅₀ value than Shilajit but 3.5 times less than 3-OH-DBP and 3 times less than 3,8 (OH)₂ DBP. Donepezil was used as reference standard which IC₅₀ value approximately 12 times more effective than 50:10:50. The results show that shilajit interferes with anti-cholinesterase activity of DBP's.

### Example 5

In another embodiment, the objective of this study is to assess the in-vitro synergistic effect of Shilajit with DBP's (Urolithins A and B) in various combinations on Rat Acetylcholinesterase-induced β-Amyloid Aggregation by using the ThT (Thioflavin-T) method. Chemical reagents and starting materials were employed as above.

Inhibition of AChE-Induced β-Amyloid Peptide Aggregation Assay

Aβ₁₋₄₀, a disordered peptide, folds into an ordered β sheet structure and further self assembles to form toxic fibrillar aggregates. These fibrillar aggregates play a crucial role in initiating the symptoms observed in AD. Inhibiting the formation of fibrillar aggregates (i.e., fibrillization) is considered as one of the most prominent approaches to develop effective therapeutics agents for AD.

The effect of 3-OH-DBP (Urolithin B: B) and 3,8-(OH)₂-DBP (Urolithin A: A) on the aggregation propensity of Aβ₁₋₄₀ and (Thiofavin-T) ThT assay measurements were performed. ThT is a molecular dye that shows maximal fluorescence when bound to fully formed Aβ₁₋₄₀ aggregates. Therefore, ThT assay was performed to study the effect of 3-(OH)-DBP (B) and 3,8-(OH)₂-DBP (A) on the fibrillization of Aβ₁₋₄₀.

Test compounds were dissolved in DMSO (5%) and diluted further with 0.215 M sodium phosphate buffer (pH 7.4). The Aβ₁₋₄₀ (10 μM and 30 μM) was incubated alone and independently with different concentrations of test compounds in the ratios shown in Tables 6 and 7, respectively.

Table 6 shows combination ratios of Aβ₁₋₄₀ (10 μM) with DBP's and shilajit with DBP's as follows.

TABLE 6

| Sample No. | Test compound | Concentration ratio (μM) |
|---|---|---|
| 1 | Aβ₁₋₄₀ | 10 |
| 2 | Aβ₁₋₄₀:Donepezil | 10:50 |
| 3 | Aβ₁₋₄₀:Shilajit | 10:50 |
| 4 | Aβ₁₋₄₀:B | 10:50 |
| 5 | Aβ₁₋₄₀:A | 10:50 |
| 6 | Aβ₁₋₄₀:(B:A) | 10:(10:50) |
| 7 | Aβ₁₋₄₀:(B:A) | 10:(30:50) |
| 8 | Aβ₁₋₄₀:(B:A) | 10:(50:50) |
| 9 | Aβ₁₋₄₀:(S:B:A) | 10:(50:10:50) |
| 10 | Aβ₁₋₄₀:(S:B:A) | 10:(50:50:50) |

Where, B = 3-(OH)-DBP A = 3,8-(OH)₂-DBP, S = Shilajit

Table 7 shows combination ratios of Aβ₁₋₄₀ (30 μM) with DBP's and shilajit with DBP's as follows.

TABLE 7

| Sample No | Test compound | Concentration ratio (μM) |
|---|---|---|
| 1 | Aβ₁₋₄₀ | 30 |
| 2 | Aβ₁₋₄₀:Donepezil | 30:50 |
| 3 | Aβ₁₋₄₀:Shilajit | 30:50 |
| 4 | Aβ₁₋₄₀:B | 30:50 |
| 5 | Aβ₁₋₄₀:A | 30:50 |
| 6 | Aβ₁₋₄₀:(B:A) | 30:(10:50) |
| 7 | Aβ₁₋₄₀:(B:A) | 30:(30:50) |
| 8 | Aβ₁₋₄₀:(B:A) | 30:(50:50) |
| 9 | Aβ₁₋₄₀:(S:B:A) | 30:(50:10:50) |
| 10 | Aβ₁₋₄₀:(S:B:A) | 30:(50:50:50) |

Where, B = 3-(OH)-DBP A = 3,8-(OH)₂-DBP, S = Shilajit

2 μL of different concentrations of Aβ₄₀ (10 and 30 μM) was incubated with 16 μL of AChE in the presence of different ratio of test compounds to obtain final concentrations of 30 or 10 μM Aβ₄₀, 230 μM AChE, 10, 20, 50 μM test compounds and 2 μL assay buffer (volume 22 μL). The mixture was incubated at room temperature for 24 or 48 hr in dark (Time period may be changed according to experiment).

Fluorescence intensities were obtained for Aβ₄₀ aggregation in the presence of inhibitors, in the absence of inhibitors and the blanks or solvent control. After incubation, 178 μL of 20 μM ThT was added. Fluorescence intensity of the solution was read at 442 or 450 nm excitation and 490 or 483 nm emission wavelengths (Total vol. 200 μL).

The percentage inhibition of the AChE-induced Aβ₁₋₄₀ aggregation was calculated as follows.

$$\% aggregation = 100 - \{ \frac{( IF l )}{( IFo )} \times 100 \} \qquad Equation ( 2 )$$

Where—IFi and IFo are fluorescence intensity obtained for the Aβ plus AChE in presence and absence of inhibitor respectively (Shidore, M., et al., “Benzylpiperidine-linked diarylthiazoles as potential anti Alzheimer agents: Synthesis and biological Evaluation,” J. Med. Chem. (2016) 59:5823-5846).

Preparation of ThT solution (100 μM): 0.32 mg of ThT was dissolved in 10 ml of ultrapure H₂O until a particulate-free solution is achieved. The solution was filtered using a 0.22-μm PES syringe filter.

Preparation of Aβ₄₀Stock: Aβ40 peptide (0.25 mg) (Calbiochem, Merck) was dissolved in hexafluoro-2-propanol (HFIP, 0.2 mL) and incubated at room temperature for 1 h. HFIP was then removed by the flow of nitrogen and further dried under vacuum. The concentration of Aβ40 peptide was determined by UV-vis spectrometry (Biotek USA; Plate reader) using a molar extinction coefficient of 1450 cm⁻¹ M⁻¹ at 276 nm. HFIP-treated A1340 was then dissolved in 10 mM PBS buffer to a concentration of 200 mM at pH 7.4. The assay was performed in triplicate and in three independent runs.

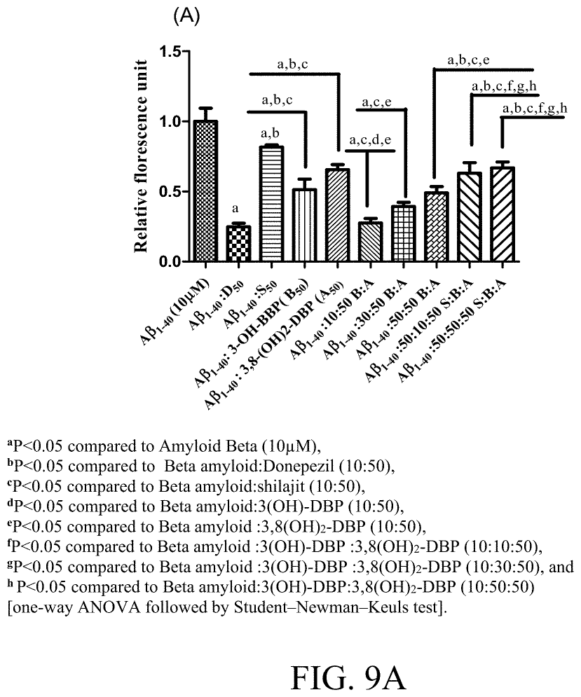

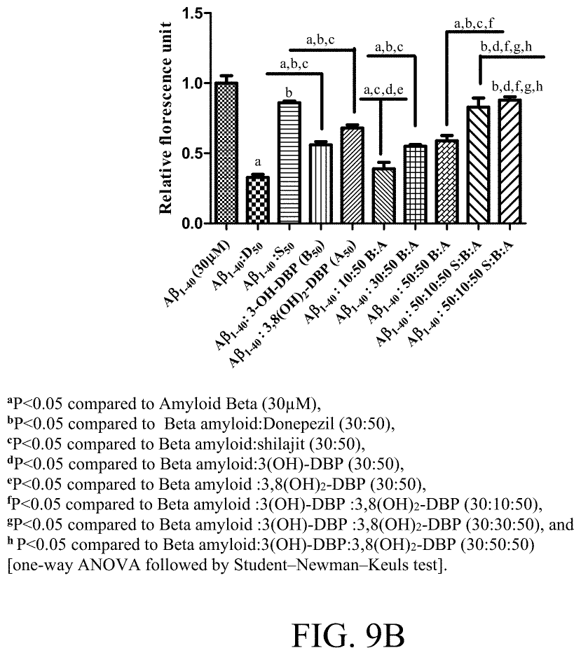

Donepezil, Shilajit, 3-OH-DBP (Urolithin B: B) and 3,8-(OH)₂-DBP (Urolithin A: A), inhibited amyloid beta (10 μM) aggregation approximately by 79%, 21%, 50% and 35% respectively. Next, the effect of varying concentrations of the 3-OH-DBP (B) (10, 30 and 50 μM) and fixed concentration of 3,8-(OH)₂-DBP (A) (50 μM) on the aggregation of Aβ40 (10 μM) were studied. The combination 10:50 (B:A) has a potent effect on Aβ40 aggregation. The 10:50 (B:A) concentration showed decrease in ThT fluorescence, corresponding to 72% inhibition of Aβ40 aggregation which was 22% and 38% higher than the 3-OH-DBP (B) and 3,8-(OH)₂-DBP (A) respectively; but 7% lower than standard drug Donepezil. On the other hand, 60% of Aβ40 aggregation was inhibited by 30:50 (B:A), which was approximately 10% and 20% higher than the 3-OH-DBP (B) and 3,8-(OH)₂-DBP (A) respectively; but 20% lower than Donepezil. Further, addition of shilajit to combination of DBP'S was studied. The addition of Shilajit decreased the percentage inhibition of amyloid beta aggregation by combination of DBP's (FIG. 9A).

Further, the above concentration ratios of test compound were incubated with fixed concentration of Aβ40 (30 μM). Donepezil, Shilajit, 3-OH-DBP (B) and 3,8-(OH)₂-DBP (A), inhibited amyloid beta aggregation approx. 65%, 10%, 41% and 32% respectively. The 10:50 (B:A) concentration showed higher effectiveness in reducing ThT Fluorescence, corresponding to 58% inhibition of Aβ40 aggregation which was 17% and 26% higher than the 3-OH-DBP (B) and 3,8-(OH)₂-DBP (A), respectively, but 7% lower than standard Donepezil. As above, the addition of Shilajit decreased the inhibition by DBP's (FIG. 9B).

These data revealed that the combination of test compound 3-OH-DBP (B) and 3,8-(OH)₂ DBP (A) at fixed ratios show synergistic effects in inhibiting of amyloid beta (10 μM, 30 μM) aggregation.

The above study shows that the 10:50 μM (Urolithin B: Urolithin A) concentration shows higher synergistic effect in amyloid beta aggregation assay by ThT method. So this combination was used for further study with Human cholinesterase enzyme assay.

### Example 6

In another embodiment, the objective of this study is to assess the in-vitro synergistic effect of DBP's (Urolithins A and B) and Shilajit with DBP's in various combinations on Recombinant Human Acetylcholinesterase by using the Ellman (1961) method.

In-vitro human recombinant AChE inhibition assay: Procedure described by Ellman et al. was used after some modification for AChE inhibition assay. Human recombinant AChE was purchased from Sigma Aldrich (St. Louis, Mo.). Acetylthiocholine iodide (ATCI), 5,5′-dithiobis (2-nitrobenzoic acid) (DTNB-Ellman's reagent) were purchased from Himedia Pvt. Ltd. (New Delhi, India). Donepezil was used as positive control groups. Tris-HCl buffer (pH 8) was used to perform the in-vitro assay. Percentage inhibition was determined at 50 mg/ml to decide the concentration range for IC₅₀ assay. Different combinations were prepared for test compound B (3-OH-DBP): test compound A (3,8-(OH)₂-DBP).

Various concentrations of test compound B (10, 30, 50 mg/ml) with a fixed concentration of test compound A and Shilajit at 50 mg/ml (shown in Tables 8 and 9, respectively) were used to determine the IC₅₀. 50 μL of human recombinant AChE (0.22 U mL⁻¹) and 10 μL of test or standard compounds were incubated in 96 well plate for 30 min. at room temperature.

Table 8 shows combination ratios of DBP's (Urolithin B:Urolithin A) as follows.

TABLE 8

| Test compound B (in mg/ml) | Test compound A (in mg/ml) |
|---|---|
| 10 | 50 |
| 30 | 50 |
| 50 | 50 |

Table 9 shows combination ratios of Shilajit with DBP's (Urolithin B:Urolithin A) as follows.

TABLE 9

| Shilajit | Test compound B | Test compound A |
|---|---|---|
| (mg/ml) | (in mg/ml) | (in mg/ml) |
| 50 | 10 | 50 |
| 50 | 30 | 50 |
| 50 | 50 | 50 |

Further, substrate i.e. ATCI (15 mM, 30 μL) was added and it was incubated for additional 30 min. Finally 160 μL (1.5 mM) of DTNB was added and the absorbance was recorded at 415 nm using Epoch microplate reader (BioTek). The IC₅₀ value was calculated using absorbance obtained from the test and standard compounds. The assay was performed in triplicate and in three independent runs (see Equation 1).

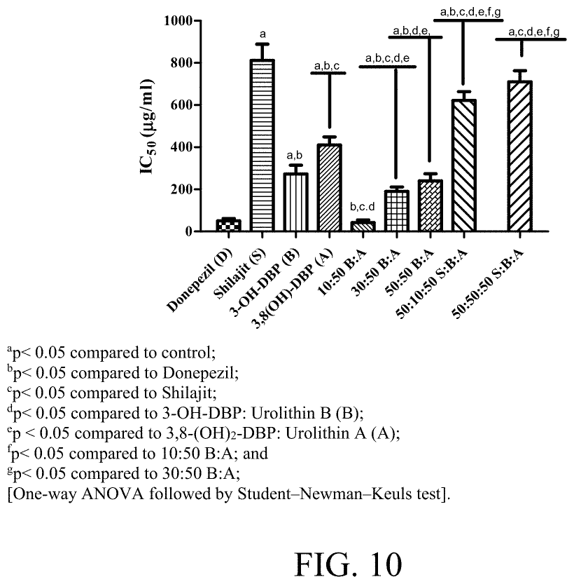

The effect of varying concentrations of Urolithin B (10, 30 and 50 mg/ml) and a fixed concentration of Urolithin A (50 mg/ml) on in-vitro AChE activity by Ellman method were studied (FIG. 10 and Table 10). Statistical analysis showed that there were significant differences among groups [F (9, 29)=54.93 p<0.05]. The IC₅₀ value of combination of 3-OH-DBP (B) and 3,8-(OH)₂-DBP (A); 10:50 (B:A) was statistically different when compared with 30:50 (B:A), 50:50 (B:A), 50:10:50 (Shilajit: Urolithin B: Urolithin A), 50:50:50 (Shilajit: Urolithin B: Urolithin A), respectively. The combination drug 10:50 (B:A) had a lower IC₅₀ value. Further, there were no significant differences between IC₅₀ values of 10:50 (B:A) and standard Donepezil.

Table 10 shows Acetylcholinesterase inhibitory activities (IC₅₀) of Urolithin A, Urolithin B, and combinations thereof.

TABLE 10

| Sample No. | Concentration ratio | IC₅₀ (μg/ml) |
|---|---|---|
| 1 | Control | 0.00 ± 0.00 |
| 2 | Donepezil | 50.48 ± 10.56 |
| 3 | 3-OH-DBP (B) | 272.67 ± 42.11 |
| 4 | 3-8-(OH)₂-DBP (A) | 410.50 ± 38.52 |
| 5 | Shilajit | 812.00 ± 77.09 |
| 6 | 10:50 (B:.A) | 42.29 ± 12.14 |
| 7 | 30:50 (B:.A) | 190.50 ± 20.58 |
| 8 | 50:50 (B:.A) | 240.33 ± 33.11 |
| 9 | 50:10:50 (S:B:A) | 622.00 ± 42.00 |
| 10 | 50:50:50 (S:B:A) | 710.00 ± 53.00 |

Where, B = 3-(OH)-DBP A = 3,8-(OH)₂-DBP, S = Shilajit

In conclusion, combination dose 10:50 (B:A) shows approximately 10 times more effective IC₅₀ value than 3,8 (OH)₂ DBP (A) and 7 times more effective than 3-OH-DBP (B); while 30:50 (B:A) is approximately 2 and 1.5 times more effective than 3,8 (OH)₂ DBP (A) and 3-OH-DBP (B), respectively. The IC₅₀ value of 50:50 (B:A) and 30:50 (B:A) were approximately the same. The effect of Donepezil was approximately was similar to that 10:50 (B:A). Shilajit showed less effective IC₅₀ value compared to DBPs and their combination. These data revealed that the 3-OH-DBP (B) and 3,8 (OH)₂ DBP (A) combination shows higher synergistic effects at 10:50 (B:A) concentration. So this concentration was taken for further study with Amplex Red kit method by using Human recombinant AChE.

### Example 7

In another embodiment, the objective of this study is to assess the in-vitro synergistic effect of DBP's (Urolithins A and B) and Shilajit with DBP's in various combinations on Recombinant Human Acetylcholinesterase by using the Amplex Red—fluorescence kit method. Human recombinant AChE was procured from Sigma Aldrich (St. Louis, Mo., USA). Amplex red kit was procured from Thermo-Fisher Scientific India, Ref no: A12217. All other chemicals and reagents were available commercially from local suppliers (Merck Pvt. Ltd., New Delhi and HiMedia Laboratories Pvt Ltd., New Delhi, India) and were of analytical grade.

In-vitro human recombinant AChE inhibition assay by Amplex Red kit method: The anticholinesterase activity of experimental drugs was measured by using Amplex red assay kit (Molecular Probes, Inc., USA). Fluorescence assay used for the screen of Human AChE activity in the presence of each experimental drug from a commercially available kit (Amplex Red, Invitrogen, Thermo Fisher Scientific, Waltham, Mass.). Acetylcholine, the product of AChE catalysis, was converted to a fluorescent called resorufin by the addition of a series of kit enzymes, including Choline oxidase (CO), horseradish peroxidase (HRP), and the fluorogenic substrate Amplex Red. As the fluorescent molecule resorufin is dependent on acetylcholine production and acetylcholine production is the product of AChE catalysis, the rate of change in fluorescence is a measurement of AChE activity. Potent inhibitors of AChE would significantly decrease the fluorescence compared to controls without inhibitor. Briefly, all reagents were prepared according the commercially available kit (Amplex Red, Invitrogen). The following procedure is designed for use with a fluorescence multi-well plate scanner. The assay was carried out in a 96-well plate, with 160 μL of total liquid volume per well. 96 wells were carried out under experimental conditions, where 40 μL of Amplex Red solution (400 μM Amplex Red solution containing 2 U/mL HRP, 0.2 U/mL choline oxidase by adding 200 μL of Amplex Red reagent stock solution, 100 μL of the HRP stock solution, 100 μL of choline oxidase (CO) stock solution and to 9.6 mL of 1× Reaction Buffer), 80 μL of Human recombinant AChE (0.22 U/ml) and 1.6 μL of varied concentrations of Urolithin B (10, 30, 50 mg/ml) with fixed concentrations of Urolithin A at 50 mg/ml, and optionally with Shilajit at 50 mg/ml, were used to determine the IC₅₀ (as shown in Tables 8 and 9 above, respectively). Further, 40 μL of 40 μM substrate acetylcholine solution were pipetted into each well.

Positive control wells, representing 100% Human recombinant AChE activity (0% human-AChE inhibition), contained, Acetylcholine, and Amplex Red solution. Negative control wells contained Acetylcholine and Amplex Red solution. Additionally, Amplex Red solution only controls were used as an additional negative control. In control wells, where necessary, 1× reaction buffer was added to bring wells to the standard volume. Fluorescence measurements were taken at baseline (immediately after substrate was added to each well), and after a 60-minute incubation at 37° C. The fluorescence micro-plate reader was measured using excitation in the range of 530-560 nm and emission detected at 590 nm. To calculate percent inhibition of AChE, CO, and HRP, the average negative control fluorescence was subtracted from the experimental group fluorescence and divided by the average positive control fluorescence. Subtracting percent activity from 100% gave percent inhibition of Human AChE.

Table 11 shows human recombinant Acetylcholinesterase inhibitory activities (IC₅₀) of Urolithin A, Urolithin B, and combinations thereof.

TABLE 11

| Sample No. | Concentration ratio | IC₅₀ (μg/ml) |
|---|---|---|
| 1 | Control | 0.00 ± 0.00 |
| 2 | Donepezil | 82.80 ± 25.86 |
| 3 | 3-OH-DBP (B) | 412.00 ± 28.28 |
| 4 | 3-8-(OH)₂-DBP (A) | 528.00 ± 70.71 |
| 5 | Shilajit | 876.13 ± 47.23 |
| 6 | 10:50 (B:.A) | 74.33 ± 21.14 |
| 7 | 30:50 (B:.A) | 284.40 ± 52.46 |
| 8 | 50:50 (B:.A) | 358.00 ± 50.74 |
| 9 | 50:10:50 (S:B:A) | 710 ± 75.30 |
| 10 | 50:50:50 (S:B:A) | 750.00 ± 60.17 |

Where, B = 3-(OH)-DBP A = 3,8-(OH)₂-DBP, S = Shilajit

As shown in Table 11 above, combination dose 10:50 (B:A) shows approximately 7 times more effective IC₅₀ value than 3,8-(OH)₂-DBP (A) and 6 times more effective than 3-OH-DBP (B); While 30:50 (B:A) is approximately 2 and 1.5 times more effective than 3,8-(OH)₂-DBP (A) and 3-OH-DBP (B) respectively. The IC₅₀ values of 30:50 (B:A) and 50:50 (B:A) were approximately the same. The effect of Donepezil was approximately was similar to that 10:50 (B:A). Shilajit showed less effective IC₅₀ value compared to DBP's and their combination. These data revealed that 3-OH-DBP (B) and 3,8-(OH)₂-DBP (A) combination shows higher synergistic effects at 10:50 (B:A) concentration. So this combination was used for further study with Human AChE-induced amyloid beta aggregation.

### Example 8

In another embodiment, the objective of this study is to assess the in-vitro synergistic effect of Shilajit with DBP's (Urolithins A and B) in various combinations on recombinant human Acetylcholinesterase-induced β-Amyloid Aggregation by using the ThT (Thioflavin-T) method. Chemical reagents and starting materials were employed as above.

Inhibition of human recombinant Acetylcholinesterase enzyme-induced β-Amyloid Peptide Aggregation Assay

The procedure of Example 5 was repeated, except that human recombinant AChE was used, with concentrations of test compounds in the ratios shown in Tables 6 and 7, respectively.

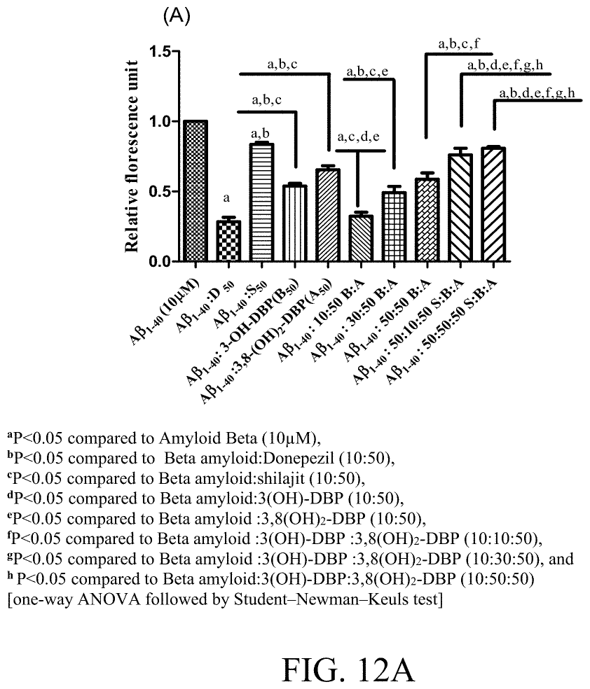

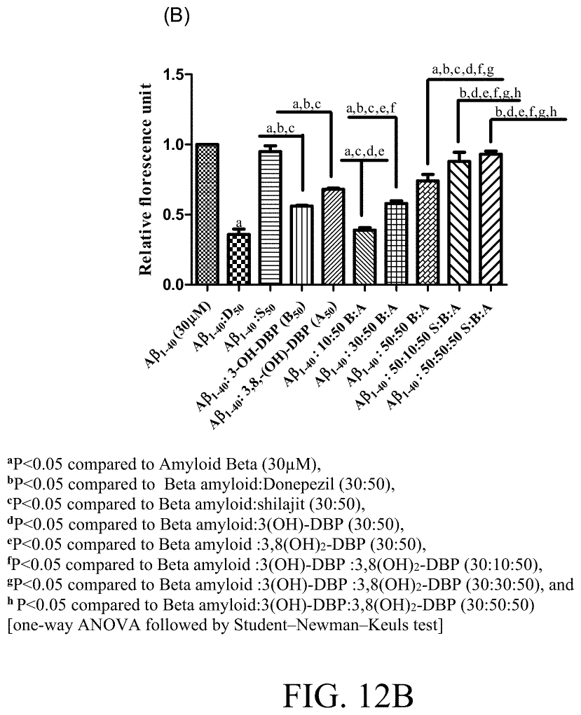

Donepezil, Shilajit, 3-OH-DBP (Urolithin B: B) and 3,8-(OH)₂-DBP (Urolithin A: A), inhibited amyloid beta (10 μM) aggregation approximately by 71%, 16%, 46% and 34% respectively. Next, the effect of varying concentrations of the 3-OH-DBP (B) (10, 30 and 50 μM) and fixed concentration of 3,8-(OH)₂-DBP (A) (50 μM) on the aggregation of Aβ40 (10 μM) were studied. The 10:50 (B:A) concentration showed a decrease in ThT Fluorescence, corresponding to 67% inhibition of Aβ40 aggregation which was 21% and 33% higher than the 3-OH-DBP (B) and 3,8-(OH)₂-DBP (A) respectively; but 4% lower than standard drug Donepezil. On the other hand, 50% of Aβ40 aggregation was inhibited by 30:50 (B:A), which was approximately 4% and 16% higher than the 3-OH-DBP (B) and 3,8-(OH)₂-DBP (A) respectively; but 21% lower than standard drug Donepezil. Further, addition of shilajit to combination of DBP's was studied. The addition of Shilajit decreased the percentage inhibition of amyloid beta aggregation by combination DBP's (FIG. 12A).

Further, the above concentration ratios of test compound were incubated with fixed concentration of Aβ40 (30 μM). Donepezil, Shilajit, 3-OH-DBP (B) and 3,8-(OH)₂-DBP (A), inhibited amyloid beta aggregation approximately 64%, 4.2%, 43% and 30% respectively. The 10:50 (B:A) concentration showed higher effectiveness in reducing ThT Fluorescence, corresponding to 60% inhibition of Aβ40 aggregation which was 17% and 30% higher than the 3-OH-DBP (B) and 3,8-(OH)₂-DBP (A), respectively; but 4% lower than standard drug Donepezil.. As above, the addition of Shilajit decreased the inhibition of DBP's (FIG. 12B).

These data revealed that only combination of test compound 3-OH-DBP (B) and 3,8-(OH)₂-DBP (A) at fixed ratios show synergistic effects in inhibition of amyloid beta (10 μM, 30 μM) aggregation.

The above study shows that the 10:50 μM (Urolithin B: Urolithin A) concentration shows higher synergistic effect in amyloid beta aggregation assay by ThT method. So this combination was used for further study with in-vivo scopolamine induced amnesic model.

### Example 9

In-Vivo Synergistic Effect of DBP's and their Combinations on Scopolamine Induced Amnesic Rat Model.

Scopolamine hydrobromide was procured from Sigma (St. Louis, Mo., USA) and Donepezil hydrochloride was obtained as a gift sample donated by Hetero Drugs Ltd, Hyderabad, India.

The experiment was conducted in accordance with the Principles of laboratory animal care. Male Wistar rats (200-250 g) were purchased from the Central Animal House, Institute of Medical Sciences, Banaras Hindu University (BHU), Varanasi, India. Experiments on animals were approved by the Institutional Animal Ethics Committee of BHU, Varanasi, India. The animals were housed in polypropylene cages under controlled environmental conditions of temperature of 25±1° C. and 45-55% relative humidity and a 12:12 h light/dark cycle. The experimental animals had free access to commercial rat feed and water ad libitum during the experiment. Animals were acclimatized for at least one week before using them for experiments and exposed only once to the experiment.

Rats were randomly allocated into eight study groups containing six in each group (n=6), as follows:

Group 1: Vehicle group: receive 0.3% CMC;

Group 2: Scopolamine group (1 mg/kg i.p.);

Group 3: Donepezil (3 mg/kg)+Scopolamine (1 mg/kg i.p) group;

Group 4: Shilajit (50 mg/kg)+Scopolamine (1 mg/kg i.p.) group;

Group 5: 3-OH-DBP (Urolithin B: B) (50 mg/kg)+Scopolamine (1 mg/kg i.p.) group;

Group 6: 3,8-(OH)₂-DBP (Urolithin A: A) (50 mg/kg) group+Scopolamine (1 mg/kg i.p.) group;

Group 7 3-OH-DBP (B) (10 mg/kg):3,8-(OH)₂-DBP (A) (50 mg/kg)+Scopolamine (1 mg/kg i.p);

Group 8: 3-OH-DBP (B) (50 mg/kg):3,8-(OH)₂-DBP (A) (50 mg/kg)+Scopolamine (1 mg/kg i.p).

Drug solution was freshly prepared in 0.3% CMC and administered by oral gavage once a day, for a period of 7 days. Briefly, the whole experiment was conducted for 8 days. Scopolamine (1 mg/kg), a muscarinic receptor antagonist, was dissolved in normal saline (0.9% NaCl) and administered intraperitoneally 1 h after drugs administration on seventh day.

Behavioral tests follow as Examples 9A and 9B.

### Example 9A

Assessment of short term memory by Y maze test paradigm. The Y-maze test was performed essentially to assess spatial recognition memory, general exploratory behavior and anxiety-like behavior by following the standard protocol (Dellu, F., Mayo, W., Cherkaoui, J., Le Moal, M., Simon, H., “A two-trial memory task with automated recording: study in young and aged rats,” Brain Res. (1992) 588: 132-139). The Y-maze apparatus consists of three identical arms (50 cm long, 16 cm wide and 32 cm high) at 120° angles to each other, radiating out from a central point. Visual cues made from colored construction paper and laboratory glassware were placed around the perimeter of the maze and above the top of the black plexiglass sides and these cues were not repeated for each test to maintain novelty to the animals. The novel arm of the Y-maze was blocked and rats were allowed to visit the other two arms of the maze for 15 min. Four hours after the first phase, animals had free access to all three arms for 5 min. The number of entries in each arm was recorded for a 5-min period. The dependent variables such as the total number of entries in all arms (for the 5 min of trial 1 and 2), the % entries in known and novel arms for the 5 min period of trial 2 and the percentage of ratio of time spent in novel arm to time spent in all the arms and in the center of the apparatus during trial 2 were measured. The total number of entries in all arms (for the 5 min of trial 1 and 2) is indicative of general exploration attitude (curiosity) and the % entries in known versus novel arm for the 5 min period of trial 2 is considered as a measure of arm discrimination (spatial recognition memory). Coping strategy or behavior to novel environment was estimated by the percentage of time spent in novel arm to time spent in all arms and in the center of the apparatus during trial 2. The decrease in the coping behavior to novel environment was considered as increase in anxiety-like behavior. An arm entry was counted when the head and two front paws were inside the arm, and duration of an arm visit was ended when the head and two front paws were outside the arm again.

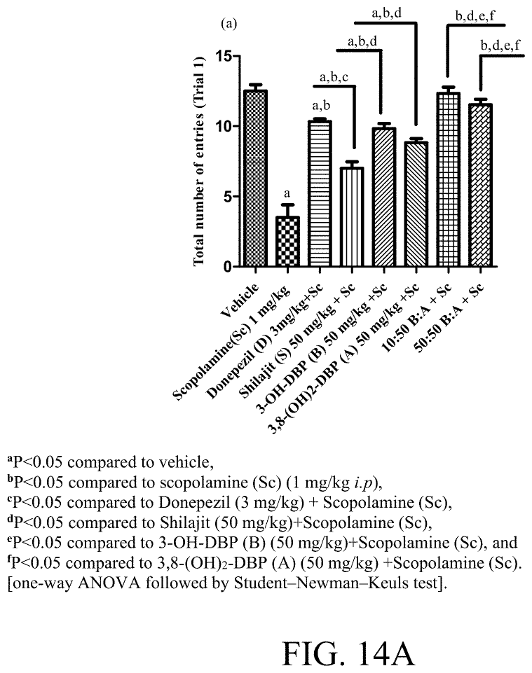

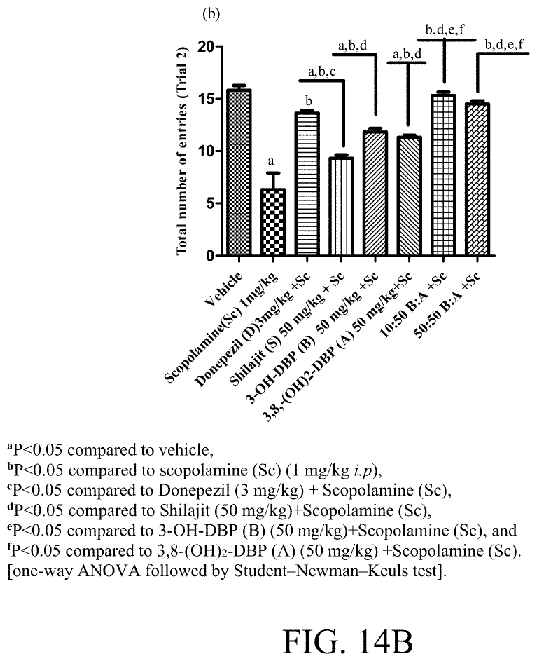

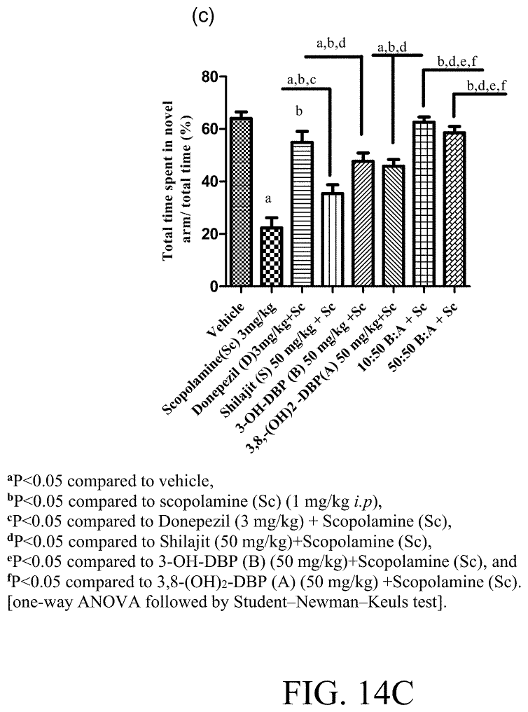

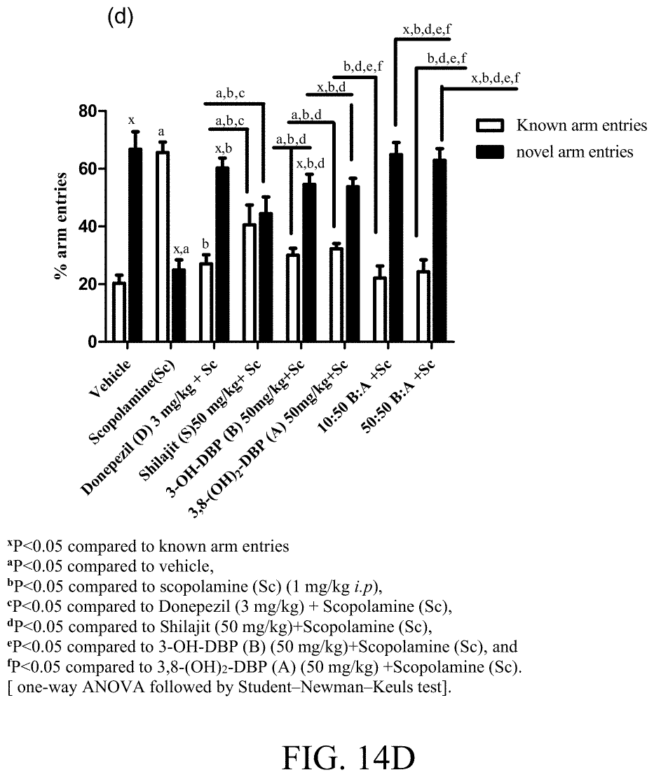

Effect of DBP's (Urolithins A and B) and their combination on Cognitive and Curiosity behavior in Y-maze Test: The effect of urolithin B: 3-OH-DBP (50 mg/kg p.o), Urolithin A: 3,8-(OH)₂-DBP (50 mg/kg p.o), Donepezil (3 mg/kg p.o), Shilajit (50 mg/kg p.o) and the combinations B:A (10:50 mg/kg p.o) and B:A (50:50 mg/kg p.o) on Scopolamine (Sc)-induced alterations in general exploratory behavior (curiosity) in trial-1 and trial-2 are depicted in FIGS. 14A and 14B, respectively. Student Newman-Keuls test suggests that scopolamine administration caused a significant decrease in curiosity behavior as compared to vehicle group rats. However, 3-OH-DBP (B) 50 mg/kg p.o and 3,8-(OH)₂-DBP (A) 50 mg/kg p.o doses attenuated the scopolamine induced decrease in curiosity behavior in trial-1 and trial-2. Notably, combinations B:A (10:50 mg/kg p.o) and B:A (50:50 mg/kg p.o) attenuated the scopolamine induced decrease in curiosity behavior in a manner similar to standard Donepezil. Additionally, DBP's in combination significantly increased curiosity behavior as compared to DBP's alone in trial-1 and trial-2. However, Shilajit 25 mg/kg b.i.d dose did not efficiently attenuate scopolamine induced decrease in curiosity behavior in trial-1 and trial-2.

Effect of DBP's and their combination on coping behavior to novel environment in Y-Maze: FIG. 14C shows the coping behavior in rats in terms of percentage of time spent in the novel arm of the apparatus during trial-2. Statistical analysis revealed that there were significant difference in among groups [F (7, 47)=21.73, p<0.05]. Student Newman-Keuls test suggests that there was a significant loss of coping behavior in scopolamine treated group animals compared to vehicle group rats. However, 3-OH-DBP (B) and 3,8-(OH)₂-DBP (A) at a 50 mg/kg p.o dose attenuated the scopolamine-induced decrease in coping strategy to a novel environment but not similar to that vehicle group. Notably, combinations B:A (10:50 mg/kg p.o) and B:A (50:50 mg/kg p.o) diminished the scopolamine induced decrease in coping strategy which was similar to the vehicle and standard Donepezil groups.

Effect of DBP's and their combination on spatial recognition memory in Y-Maze Test: The synergistic effect of experimental drugs on scopolamine-induced alterations in spatial recognition memory on is depicted in FIG. 14D. Two-way ANOVA showed that there were significant difference in arm discrimination behavior during trial-2 among groups ([F (7, 80)=0.098; P<0.05]), among known and novel arms ([F (1, 80)=107; P<0.05], and a significant interaction between group and arms ([F (7, 80)=24; P<0.05], in Y-maze test paradigm. Post-hoc analysis revealed that scopolamine induced group failed to discriminate the percentage arms entries into known versus novel compared to vehicle group indicating impaired in spatial recognition memory. 3-OH-DBP (B) and 3,8-(OH)₂-DBP (A), in the dose of 50 mg/kg significantly reversed the scopolamine induced failed the discrimination behavior but not similar as to the vehicle group. Notably, combinations B:A (10:50 mg/kg p.o) and B:A (50:50 mg/kg p.o) significantly mitigated the scopolamine induced impairment in spatial recognition memory by increasing the percentage of arms entries in the novel environment. It is interesting to note that there were no significant differences between vehicle, Donepezil, and combinations B:A (10:50 mg/kg p.o) and B:A (50:50 mg/kg p.o) in both known and novel arm entries.

### Example 9B

Passive avoidance (PA) test: The PA task is a fear-aggravated test used to evaluate learning and memory in rodent models of CNS disorders. In this test, subjects learn to avoid an environment in which an aversive stimulus (such as a foot-shock) was previously delivered. The animals can freely explore the light and dark compartments of the chamber, and a mild foot shock is delivered in one side of the compartment. Animals eventually learn to avoid foot shock by not entering into dark chamber. Briefly, the step-through passive avoidance apparatus consisted of a light chamber (20 cm×20 cm×30 cm), made of transparent plastic, and a dark chamber (20 cm×20 cm×30 cm), the walls of which were made of dark opaque plastic. The floor of both chambers consisted of stainless steel rods (3 mm diameter) spaced 1 cm apart. The floor of the dark chamber could be electrified using a shock generator. A rectangular opening (6 cm×8 cm) was located between the two chambers and could be closed by an opaque, guillotine door. Thirty minutes after the administration of scopolamine or vehicle on seventh day, rats were subjected to the PA test by placing in a light compartment. After an acclimatization period of 30 s, the guillotine door was opened and closed automatically after entry of the rat into the dark compartment. The subject received a low-intensity foot shock (0.5 mA; 10 s) in the dark compartment. The transfer of the animal from one compartment to another was recorded and transfer latency time (TLT) was calculated in seconds. The duration of a trial was 270 s. The first trial was for acquisition, and retention was tested in a second trial given 24 h after the first trial. The shock was not delivered in the retention trials to avoid reacquisition. The criterion for learning was taken as an increase in the TLT on retention trials as compared to acquisition trial.

Effect of DBP's and their Combination of Passive Avoidance Task.

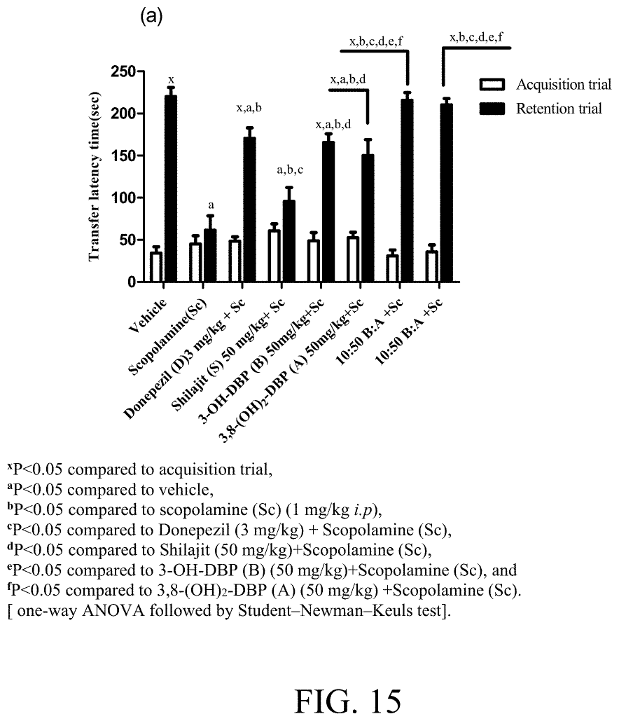

The effect of 3-OH-DBP (B) (50 mg/kg) and 3,8-(OH)₂-DBP (A) (50 mg/kg) on scopolamine induced changes in retention and recall in passive avoidance task on day 7 of the drug treatment schedule are depicted in FIG. 15. Statistical analysis by Two-way ANOVA revealed that there were significant differences in transfer latency among the treatment groups [F (7, 80)=13, p<0.05] with time [F (1, 80)=443, p<0.05] and an interaction between group and time ([F (7, 80)=18.8; P<0.05]. Post hoc analysis revealed that there were no significant differences in the transfer latency during acquisition trial (TLa) among the eight groups. But in the retention trial, a significant reduction in the transfer latency (TLr) were observed in the scopolamine treated group compared with vehicle group indicating impaired in learning and memory. Administration of scopolamine 30 min before acquisition trial caused memory impairment as shown by no significant increase in the TLT of the retention trial as compared to the acquisition trial. Treatment with 3-OH-DBP (B) and 3,8-(OH)₂-DBP (A) at a dose of 50 mg/kg for 7 days prevented scopolamine-induced dementia in rats as revealed by significant increase in TLT of retention trial in comparison to acquisition trial but less effective as compared to the vehicle treated group. Notably, combinations B:A (10:50 mg/kg p.o) and B:A (50:50 mg/kg p. o) significantly mitigated the scopolamine induced impairment in learning and memory by increase in the TLT of the retention trial as compared to the acquisition trial, which was similar to the vehicle groups. Additionally, B:A (10:50 mg/kg p.o) and B:A (50:50 mg/kg p.o) significantly increased transfer latency in the retention trial as compared to 3-OH-DBP (B), 3,8-(OH)₂-DBP (A), and standard Donepezil thus indicating synergistic effects. Further, Shilajit at a dose of 25 mg/kg b.i.d caused impairment in memory as shown by no significant increase in the TLT of the retention trial as compared to the acquisition but significant increase in the TLT in the retention trial as compared to scopolamine treated group.

Evaluation of the acetylcholinergic system follows as Examples 9C and 9D.

### Example 9C

Sample preparation: The animals were killed by decapitation and the brain tissues were dissected out in ice-cold conditions and stored at −80° C. until use. Tissue was homogenized in 1.0 ml of 0.1M perchloric acid with a Potter-Elvehjem homogenizer. The homogenate was kept in the polypropylene tubes for 15 min after which 40 μl of 4M potassium acetate was added to adjust the pH to 4.0 followed by centrifugation for 15 min at 4000×g. Supernatant was used to estimate acetylcholine and AChE activities.

Spectrofluorometric assay of acetylcholinesterase activity: The acetylcholinesterase activity in brain tissues was measured using Amplex red assay kit (Molecular Probes, Inc., USA). An acetylcholinesterase standard curve was used in each experiment. Briefly, Begin the reactions by adding 0.1 ml of control (10 μM H₂O₂) and tissue homogenate were taken in two separate polypropylene tubes and then 0.1 ml working solution of 400 μM Amplex Red reagent containing 2 U/mL HRP, 0.2 U/mL choline oxidase and 100 μM acetylcholine were added to each tube. After incubation for 45 min, the fluorescence was recorded with the help of spectrofluorometer at 530 nm excitation and 590 nm emission wavelengths. The protein content was determined using standard protocol (Lowry, O. H., Rosebrough, N.J., Farr, A. L., Randall, R. J., “Protein measurement with the Folin phenol reagent,” J. Biol. Chem. (1951) 193: 265-275).

Statistical analyses: The results were expressed as means±Standard Error mean (SEM). The intergroup variation was measured by one-way ANOVA followed by Student-Newman-Keuls test. Two-way followed by Bonferroni test was employed to discover the intergroup variation in Y maze test considering groups and arms as two independent variables. Statistical analysis was done by using Graph Pad prism version 5 (San Diego, Calif.). A level of p<0.05 was accepted as statistically significant.

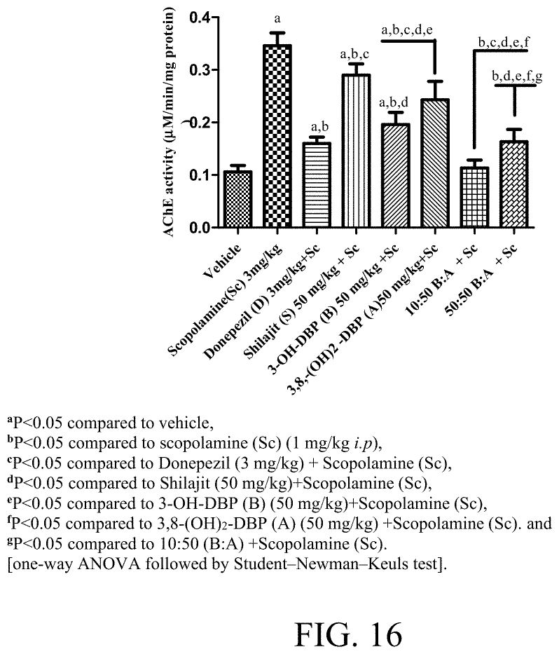

The effect of Urolithin B: 3-OH-DBP (50 mg/kg p.o), Urolithin A: 3,8-(OH)₂-DBP (50 mg/kg p.o), Donepezil (3 mg/kg p.o), Shilajit (25 mg/kg b.i.d p.o) and the combination of DBP's on scopolamine-induced alterations in acetylcholinesterase activity in HIP homogenate was tested (see FIG. 16). One-way ANOVA revealed that there were significant differences in the acetylcholinesterase activity among brain regions ([F (7, 23)=25.76, p>0.05]. Student Newman-Keuls test suggests that scopolamine administration caused a significant increase acetylcholinesterase activity compared to vehicle group. However, 3-OH-DBP (B) and 3,8-(OH)₂-DBP (A) at 50 mg/kg dose attenuated the scopolamine induced increase acetylcholinesterase activity among brain regions but statistically significant with vehicle group. Donepezil was used as reference standard which significantly reversed the scopolamine induced increased acetylcholinesterase activity and was similar that of 3-OH-DBP (B) 50 mg/kg group. 3,8-(OH)₂-DBP (A)-50 mg/kg group was less effective than 3-OH-DBP (B) 50 mg/kg. However, scopolamine induced increase in acetylcholinesterase activity was reversed by DBP's in combination and was similar to that vehicle group. Interestingly, B:A (10:50 mg/kg p.o) was found to be more effective than Donepezil. B:A (50:50 mg/kg p.o) also reversed scopolamine induced increased acetylcholinesterase activity but statistically not significant when compared with Donepezil. Additionally, Shilajit at 25 mg/kg b.i.d dose did not efficiently attenuate scopolamine induced increase acetylcholinesterase activity in HIP tissue.

### Example 9D

Samples were prepared as in Example 9C. The amount of acetylcholine in brain tissues was measured using Amplex red assay kit (Molecular Probes, Inc., USA). An acetylcholine standard curve was used in each experiment. Briefly, the reactions are initiated by adding 0.1 ml of control (10 μM H₂O₂) and tissue homogenates were taken in two separate polypropylene tubes and then 0.1 ml working solution of 400 μM Amplex Red reagent containing 2 U/mL HRP, 0.2 U/mL choline oxidase and 1 U/mL acetylcholinesterase were added to each tube. After incubation for 45 min, the fluorescence was recorded with the help of spectrofluorometer at 530 nm excitation and 590 nm emission wavelengths. The protein content was determined using standard protocol (Lowry, et al., 1951).

Statistical analyses: The results were expressed as means±Standard Error mean (SEM). The intergroup variation was measured by one-way ANOVA followed by Student-Newman-Keuls test. Two-way followed by Bonferroni test was employed to discover the intergroup variation in Y maze test considering groups and arms as two independent variables. Statistical analysis was done by using Graph Pad prism version 5 (San Diego, Calif.). A level of p<0.05 was accepted as statistically significant.

The effect of DBP's and their combination on hippocampal acetylcholine (ACh) level was measured.

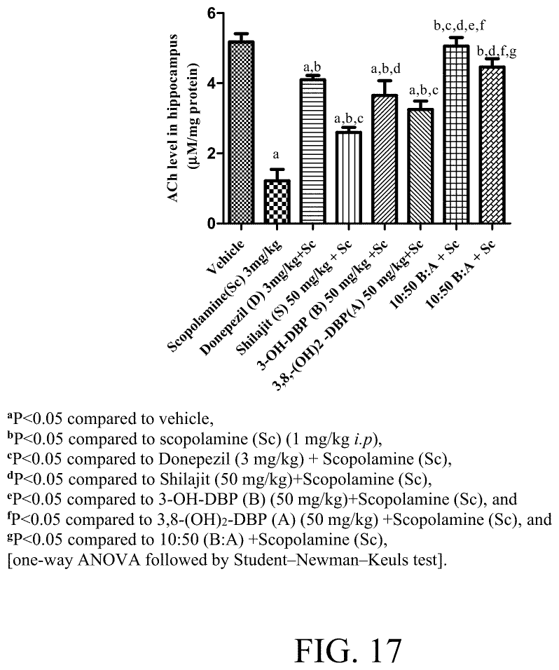

One-way ANOVA revealed that there were significant differences in the level of ACh among brain regions ([F (7, 23)=25.76, p>0.05]. Student Newman-Keuls test suggests that scopolamine administration caused a significant decrease in ACh level as compare to vehicle group rat (see FIG. 17). However, 3-OH-DBP (B) (50 mg/kg) and 3,8-(OH)₂-DBP (A) (50 mg/kg) doses attenuated the scopolamine induced decrease in ACh level among brain regions but was statistically significant with vehicle group. The ACh level based on Donepezil was the same as 3-OH-DBP (B) (50 mg/kg) group. A significant difference was observed between Donepezil treatment and the 3,8-(OH)₂-DBP (A) 50 mg/kg group. However, scopolamine-induced decrease in ACh level was significantly reversed by combinations B:A (10:50 mg/kg p.o) and B:A (50:50 mg/kg p.o) which was similar to Donepezil and vehicle groups. Additionally, the Shilajit 50 mg/kg dose did not efficiently attenuate the scopolamine induced decrease in ACh level in HIP tissue.

Previous studies have demonstrated that scopolamine (1 mg/kg i.p) causes brain dysfunction and an impairment of learning and memory (Ohja, R., et al., 2010). Herein it was observed that scopolamine caused an impairment of memory in Y-maze task and passive avoidance task, both of which are considered to involve spatial and retention and recall aspects of learning and memory respectively.

Salient findings of the result showed that scopolamine caused alteration in number changes on behavior indices in the Y-maze paradigm like general exploration attitude (curiosity), coping behavior to novel environment (anxiety-like behavior) and % entries in known versus novel arms in trial 2 (spatial recognition memory) to resolve the task (Joshi, R., et al. “Silibinin ameliorates LPS-induced memory deficits in experimental animals,” Neurobiology of learning and memory (2014) December; 116:117-313; and Tripathi, A., et al., 2017). These results indicate that scopolamine has decreased the curiosity behavior indicating impairment of short term memory. 3-OH-DBP (B) (50 mg/kg) and 3,8-(OH)₂-DBP (A) (50 mg/kg) attenuated the scopolamine induced decrease in curiosity behavior in trial-1 and trial-2. Notably, DBP's combinations B:A (10:50 mg/kg p.o) and B:A (50:50 mg/kg p.o) mitigated the scopolamine induced decrease in curiosity behavior in trial-1 and trial-2, which was similar to Donepezil. Additionally, Shilajit 50 mg/kg did change the curiosity behavior but not as significantly as the combination of DBP's.

Further, scopolamine decreased coping strategy to the novel environment in the Y-maze task and shortened transfer latency during retention trial in the passive avoidance test. These results revealed that scopolamine caused increase in anxiety-like behavior (Y maze task) and impaired retention and recall capability or ability to learn in the passive avoidance task.

Shilajit altered the coping behavior and transfer latency but was not as efficient as Donepezil. 3-OH-DBP (B) (50 mg/kg) and 3,8-(OH)₂-DBP (A) (50 mg/kg) reversed the scopolamine-induced decreased coping behavior and transfer latency (retention trial) significantly better than shilajit. However, most notably, DBP's in combinations mitigated significantly the scopolamine-induced decrease in coping behavior and transfer latency compared to shilajit and individual DBP's. One more intrusting fact noted during the study was that passive avoidance response can be affected by pain threshold or fear. In addition, it was also known that the lesion in the septum causes impairment in fear condition and an increase in defensiveness and attack. These findings point out the possibility that scopolamine induced impairment in passive avoidance responses was due to the change in pain threshold or fear.

Furthermore, it was determined that scopolamine induced a decrease in spatial recognition memory in the Y-maze task. These findings revealed that scopolamine decreased in arm discrimination in trial-2 indicating impairment of short term memory. 3-OH-DBP (B) and 3,8-(OH)₂-DBP (A), at a dose of 50 mg/kg reversed the scopolamine induced decreased spatial recognition memory. And most notably, DBP's in combinations significantly mitigated the scopolamine induced impairment in spatial recognition memory by increasing the percentage of arms entries in the novel environment.

It is interesting to be note that combinations B:A (10:50 mg/kg p.o) and B:A (50:50 mg/kg p.o) demonstrated synergistic effects by significantly increasing spatial recognition memory as compared to 3-OH-DBP (B) (50 mg/kg p.o) and 3,8-(OH)₂-DBP(A) (50 mg/kg p.o).

Acetylcholine is widely considered as the most important neurotransmitter involved in the regulation of cognitive functions. This is the major reason for the use of AChE inhibitors for the symptomatic treatment of disease related to impaired cognition, e.g., Alzheimer's disease in its early stages. There is extensive evidence linking the central cholinergic system to memory. Cognitive dysfunction has been shown to be associated with impaired cholinergic function and conversely facilitation of central cholinergic activity with improved memory. Increased acetylcholinesterase activity also generally results in a diminished ability to learn and form new memories (Meena, J., et al., “Asparagus racemosus competitively inhibits in vitro the acetylcholine and monoamine metabolizing enzymes,” Neurosci. Lett. (2011) 503(1): 6-9). Therefore, the activity of these enzymes was evaluated in the hippocampus of treated rats. In the study, scopolamine increased AChE activity in the hippocampus that is inversely related to the ACh concentration. 3-OH-DBP (Urolithin B) and 3,8-(OH)₂-DBP (Urolithin A) attenuated the scopolamine induced increase of acetylcholinesterase activity and resulting decrease in ACh levels. Most notably, combinations B:A (10:50 wt/wt) and B:A (50:50 wt/wt) mitigated the scopolamine induced increase acetylcholinesterase activity and decrease in ACh level significantly better than either B or A, separately. Moreover, Shilajit, individual DBP's were measured as equipotent to Donepezil.

In summary: The combination of 3-OH-DBP (B) and 3,8-(OH)₂-DBP (A) (10:50 wt/wt) showed in vitro synergistic acetylcholinesterase activity. The same effect was observed with human acetylcholinesterase enzyme thus underscoring the clinical potential of the observed activity.

Combination of 3-OH-DBP (B) and 3,8-(OH)₂-DBP (A) (10:50 wt/wt) showed in vitro synergistic activity in inhibiting aggregation of β-amyloid.

Administration of scopolamine in animals produces short-term learning and memory deficits, which are considered to be characteristic of cholinergic dysfunction in AD. Therefore, the scopolamine-induced amnesic model has been widely accepted as a pharmacological model of cognitive impairments useful for screening potential cognitive-enhancing agents.

Combination of 3-OH-DBP (B) and 3,8-(OH)₂-DBP (A) (10:50 wt/wt) showed in vivo attenuation of several parameters in the scopolamine induced amnesia model in rats.

The total number of entries in all arms (trial 1 and 2) is indicative of general exploration attitude (curiosity) which was decreased by scopolamine. This effect was attenuated by the combination of 3-OH-DBP (B) and 3,8-(OH)₂-DBP (A) (10:50 wt/wt). This indicates that this combination significantly mitigated the scopolamine induced impairment in spatial recognition memory by increasing the percentage of arms entries in the novel environment. The % entries in known versus novel arms for the 5 min period of trial 2 is considered as a measure of arm discrimination (spatial recognition memory). There was decrease in spatial memory by scopolamine which was reversed by the combination of 3-OH-DBP (B) and 3,8-(OH)₂-DBP (A) (10:50 wt/wt).

Coping strategy or behavior in a novel environment was estimated by the percentage of time spent in novel arm to time spent in all arms and in the center of the apparatus during trial 2. The decrease in the coping behavior in a novel environment was considered as increase in anxiety-like behavior. Thus the combination of 3-OH-DBP (B) and 3,8-(OH)₂-DBP (A) (10:50 wt/wt) showed anxiolytic activity by increasing coping behavior.

Therefore, in the Examples described above, combinations B:A (10:50 wt/wt) and B:A (50:50 wt/wt) showed synergistic effects to ameliorate short term memory, spatial memory deficits, impaired ability to learn and recall in the rats which had received Scopolamine (Sc) injection.

The activity at the lower combination dose of B:A (10:50 wt/wt) showed approx. equipotent effects when compared to Donepezil.

Mechanistically, decreased acetylcholine levels generally results in a diminished ability to learn and form new memories. The concentration of acetylcholine in the brain is dynamically regulated by the activity of AChE. AChE hydrolyzes acetylcholine into acetic acid and Choline, and consequently AChE inhibitors prolong the action of acetylcholine synapses and enhance cholinergic neurotransmission.

Therefore, but without being bound by any theory, the AChE activity of these enzymes was evaluated in the hippocampus. Scopolamine increased AChE activity in the hippocampus and decreased ACh concentration.

Therefore, as shown in the Examples described above, combinations 3-OH-DBP (B): 3,8-(OH)₂-DBP (A) (10:50 wt/wt) and 3-OH-DBP (B): 3,8-(OH)₂-DBP (A) (50:50 wt/wt) decreased AChE activity and increased in ACh level in rats which had received the scopolamine injection.

The lower combination dose of B:A (10:50 wt/wt) showed greater synergistic effects which were as effective as Donepezil.

These results suggest that the synergistic effects of experimental drugs in the scopolamine-induced-amnesic rat model may solely result from direct inhibition of AChE that is inversely correlated with increases in acetylcholine concentration.

The combination of B:A (10:50 wt/wt) showed better synergistic activity than other ratios. The pharmacological effects were more potent than shilajit, 3-OH-DBP (B) and 3,8-(OH)₂-DBP (A), separately. In most cases the combination was equipotent with donepezil.

The nutraceutical compositions of the present invention may be administered in combination with a nutraceutically acceptable carrier. The active ingredients in such formulations may comprise from 1% by weight to 99% by weight, or alternatively, 0.1% by weight to 99.9% by weight. “Nutraceutically acceptable carrier” means any carrier, diluent or excipient that is compatible with the other ingredients of the formulation and not deleterious to the user. In accordance with one embodiment, suitable nutraceutically acceptable carriers can include ethanol, aqueous ethanol mixtures, water, fruit and/or vegetable juices, and combinations thereof.

The pharmaceutical compositions of the present invention may be administered in combination with a pharmaceutically acceptable carrier. The active ingredients in such formulations may comprise from 1% by weight to 99% by weight, or alternatively, 0.1% by weight to 99.9% by weight. “Pharmaceutically acceptable carrier” means any carrier, diluent or excipient that is compatible with the other ingredients of the formulation and not deleterious to the user.

Delivery System

Suitable dosage forms include tablets, capsules, solutions, suspensions, powders, gums, and confectionaries. Sublingual delivery systems include, but are not limited to, dissolvable tabs under and on the tongue, liquid drops, and beverages. Edible films, hydrophilic polymers, oral dissolvable films or oral dissolvable strips can be used. Other useful delivery systems comprise oral or nasal sprays or inhalers, and the like.

For oral administration, shilajit, Urolithin A, Urolithin B, or combinations thereof, may be further combined with one or more solid inactive ingredients for the preparation of tablets, capsules, pills, powders, granules or other suitable dosage forms. For example, the active agent may be combined with at least one excipient such as fillers, binders, humectants, disintegrating agents, solution retarders, absorption accelerators, wetting agents, absorbents, or lubricating agents. Other useful excipients include magnesium stearate, calcium stearate, mannitol, xylitol, sweeteners, starch, carboxymethylcellulose, microcrystalline cellulose, silica, gelatin, silicon dioxide, and the like.

The components of the invention, together with a conventional adjuvant, carrier, or diluent, may thus be placed into the form of pharmaceutical compositions and unit dosages thereof. Such forms include solids, and in particular tablets, filled capsules, powder and pellet forms, and liquids, in particular aqueous or non-aqueous solutions, suspensions, emulsions, elixirs, and capsules filled with the same, all for oral use, suppositories for rectal administration, and sterile injectable solutions for parenteral use. Such pharmaceutical compositions and unit dosage forms thereof many comprise conventional ingredients in conventional proportions, with or without additional active compounds or principles, and such unit dosage forms may contain any suitable effective amount of the active ingredient commensurate with the intended daily dosage range to be employed.

The components of the present invention can be administered in a wide variety of oral and parenteral dosage forms. It will be obvious to those skilled in the art that the following dosage forms may comprise, as the active component, either a chemical compound of the invention or a pharmaceutically acceptable salt of a chemical compound of the invention.

For preparing pharmaceutical compositions from a chemical compound of the present invention, pharmaceutically acceptable carriers can be either solid or liquid. Solid form preparations include powders, tablets, pills, capsules, cachets, suppositories, and dispersible granules. A solid carrier can be one or more substances which may also act as diluents, flavoring agents, solubilizers, lubricants, suspending agents, binders, preservatives, tablet disintegrating agents, or an encapsulating material.

In powders, the carrier is a finely divided solid, which is in a mixture with the finely divided active component. In tablets, the active component is mixed with the carrier having the necessary binding capacity in suitable proportions and compacted in the shape and size desired.

The powders and tablets preferably contain from five or ten to about seventy percent of the active compound(s). Suitable carriers are magnesium carbonate, magnesium state, talc, sugar, lactose, pectin, dextrin, starch, gelatin, tragacanth, methylcellulose, sodium carboxymethlycellulose, a low melting wax, cocoa butter, and the like. The term “preparation” is intended to include the formulation of the active compound with encapsulating material as carrier providing a capsule in which the active component, with or without carriers, is surrounded by a carrier, which is thus in association with it. Similarly, cachets and lozenges are included. Tablets, powders, capsules, pills, cachets, and lozenges are included. Tablets, powders, capsules, pills, cachets, and lozenges can be used as solid forms suitable for oral administration.

Liquid preparations include solutions, suspensions, and emulsions, for example, water or water-propylene glycol solutions. For example, parenteral injection liquid preparations can be formulated as solutions in aqueous polyethylene glycol solution. The chemical compound according to the present invention may thus be formulated for parenteral administration (e.g. by injection, for example bolus injection or continuous infusion) and may be presented in unit dose for in ampoules, pre-filled syringes, small volume infusion or in multi-dose containers with an added preservative. The compositions may take such forms as suspensions, solutions, or emulsions in oily or aqueous vehicles, and may contain formulation agents such as suspending, stabilising and/or dispersing agents. Alternatively, the active ingredient may be in powder form, obtained by aseptic isolation of sterile solid or by lyophilization from solution, for constitution with a suitable vehicle, e.g. sterile, pyrogen-free water, before use.

Aqueous solutions suitable for oral use can be prepared by dissolving the active component in water and adding suitable colorants, flavors, stabilizing and thickening agents, as desired. Aqueous suspensions suitable for oral use can be made by dispersing the finely divided active component in water with viscous material, such as natural or synthetic gums, resins, methylcellulose, sodium carboxymethylcellulose, or other well known suspending agents.

Compositions suitable for topical administration in the mouth includes lozenges comprising the active agent in a flavored base, usually sucrose and acacia or tragacanth; pastilles comprising the active ingredient in an inert base such as gelatin and glycerine or sucrose and acacia; and mouthwashes comprising the active ingredient in suitable liquid carrier.

Solutions or suspensions are applied directly to the nasal cavity by conventional means, for example with a dropper, pipette or spray. The compositions may be provided in single or multi-dose form. In compositions intended for administration to the respiratory tract, including intranasal compositions, the compound will generally have a small particle size for example of the order of 5 microns or less. Such a particle size may be obtained by means known in the art, for example by micronization.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packaged tablets, capsules, and powders in vials or ampoules. Also, the unit dosage form can be a capsule, tablet, cachet, or lozenges itself, or it can be the appropriate number of any of these in packaged form.

Tablets, capsules and lozenges for oral administration and liquids for oral use are preferred compositions. Solutions or suspensions for application to the nasal cavity or to the respiratory tract are preferred compositions. Transdermal patches for topical administration to the epidermis are preferred.

Further details on techniques for formulation and administration may be found in the latest edition of Remington's Pharmaceutical Sciences (Mack Publishing Co., Easton, Pa.).

Solid nutritional compositions for oral administration may optionally contain, in addition to the above enumerated nutritional composition ingredients or compounds: carrier materials such as corn starch, gelatin, acacia, microcrystalline cellulose, kaolin, dicalcium phosphate, calcium carbonate, sodium chloride, alginic acid, and the like; disintegrators including, microcrystalline cellulose, alginic acid, and the like; binders including acacia, methylcellulose, sodium carboxymethylcellulose, polyvinylpyrrolidone, hydroxypropyl methylcellulose, ethyl cellulose, and the like; and lubricants such as magnesium stearate, stearic acid, silicone fluid, talc, waxes, oils, colloidal silica, and the like. The usefulness of such excipients is well known in the art.

In one preferred embodiment, the nutritional composition may be in the form of a liquid. In accordance with this embodiment, a method of making a liquid composition is provided.

Liquid nutritional compositions for oral administration in connection with a method for preventing and/or treating inflammation, colds and/or flu can be prepared in water or other aqueous vehicles. In addition to the above enumerated ingredients or compounds, liquid nutritional compositions can include suspending agents such as, for example, methylcellulose, alginates, tragacanth, pectin, kelgin, carrageenan, acacia, polyvinylpyrrolidone, polyvinyl alcohol, and the like. The liquid nutritional compositions can be in the form of a solution, emulsion, syrup, gel, or elixir including or containing, together with the above enumerated ingredients or compounds, wetting agents, sweeteners, and coloring and flavoring agents. Various liquid and powder nutritional compositions can be prepared by conventional methods. Various ready-to-drink formulations (RTD's) are contemplated.

Routes of Administration

The compositions may be administered by any suitable route, including but not limited to oral, sublingual, buccal, ocular, pulmonary, rectal, and parenteral administration, or as an oral or nasal spray (e.g. inhalation of nebulized vapors, droplets, or solid particles). Parenteral administration includes, for example, intravenous, intramuscular, intraarterial, intraperitoneal, intranasal, intravaginal, intravesical (e.g., to the bladder), intradermal, transdermal, topical, or subcutaneous administration. Also contemplated within the scope of the invention is the instillation of a pharmaceutical composition in the body of the patient in a controlled formulation, with systemic or local release of the drug to occur at a later time. For example, the drug may be localized in a depot for controlled release to the circulation, or for release to a local site.

Pharmaceutical compositions of the invention may be those suitable for oral, rectal, bronchial, nasal, pulmonal, topical (including buccal and sub-lingual), transdermal, vaginal or parenteral (including cutaneous, subcutaneous, intramuscular, intraperitoneal, intravenous, intraarterial, intracerebal, intraocular injection or infusion) administration, or those in a form suitable for administration by inhalation or insufflations, including powders and liquid aerosol administration, or by sustained release systems. Suitable examples of sustained release systems include semipermeable matrices of solid hydrophobic polymers containing the compound of the invention, which matrices may be in form of shaped artices, e.g. films or microcapsules.

The use of the terms “a,” “an,” “the,” and similar referents in the context of describing the presently claimed invention (especially in the context of the claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. Use of the term “about” is intended to describe values either above or below the stated value in a range of approx. ±10%; in other embodiments the values may range in value either above or below the stated value in a range of approx. ±5%; in other embodiments the values may range in value either above or below the stated value in a range of approx. ±2%; in other embodiments the values may range in value either above or below the stated value in a range of approx. ±1%. The preceding ranges are intended to be made clear by context, and no further limitation is implied. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

While in the foregoing specification this invention has been described in relation to certain embodiments thereof, and many details have been put forth for the purpose of illustration, it will be apparent to those skilled in the art that the invention is susceptible to additional embodiments and that certain of the details described herein can be varied considerably without departing from the basic principles of the invention.

All references cited herein are incorporated by reference in their entirety. The present invention may be embodied in other specific forms without departing from the spirit or essential attributes thereof and, accordingly, reference should be made to the appended claims, rather than to the foregoing specification, as indicating the scope of the invention.

1. A composition comprising urolithin A and urolithin B, wherein the wt./wt. ratio of urolithin B to urolithin A is from about 0.2:1 to about 0.6:1.

2. The composition of claim 1, wherein the wt./wt. ratio of urolithin B to urolithin A is about 0.2:1.

3. A pharmaceutical or nutraceutical composition of claim 1, further comprising a pharmaceutically or nutraceutically acceptable carrier.

4. The composition of claim 1, wherein a therapeutically effective dosage is from about 100 mg to about 1000 mg.

5. The composition of claim 1, wherein a therapeutically effective dosage is from about 100 mg to about 500 mg.

6. The pharmaceutical composition of claim 3, for use in a method for treating or preventing a dementia-related disorder in a human subject.

7. The pharmaceutical composition of claim 6, wherein the dementia-related disorder is Alzheimer's disease.

8. The pharmaceutical composition of claim 6, wherein the composition is administered in a daily dose of from about 1.5 mg/kg to about 8.0 mg/kg.

9. The pharmaceutical composition of claim 6, wherein the composition has an inhibitory concentration (IC₅₀) of about 0.05 micro-g/ml to about 0.06 micro-g/ml for inhibition of acetylcholinesterase activity.

10. The pharmaceutical composition of claim 3, for use in a method for treating or preventing an anxiety disorder in a human subject.

11. The pharmaceutical composition of claim 10, wherein the composition is administered in a daily dose of from about 1.5 mg/kg to about 8.0 mg/kg.

12. The pharmaceutical composition of claim 10, wherein the composition has an inhibitory concentration (IC₅₀) of about 0.05 micro-g/ml to about 0.06 micro-g/ml for inhibition of acetylcholinesterase activity.

13. A method for treating or preventing a cognitive disorder in an individual, comprising administering to the individual in need of such treatment a therapeutically effective amount of a composition comprising urolithin A and urolithin B, wherein the wt./wt. ratio of urolithin B to urolithin A is from about 0.2:1 to about 0.6:1.

14. The method of claim 13, wherein the cognitive disorder is selected from the group consisting of a dementia-related disorder, stress-induced dementia, anxiety, depression, and Alzheimer's disease.

15. The method of claim 13, wherein the composition has a wt./wt. ratio of urolithin B to urolithin A is from about 0.2:1 and has an inhibitory concentration (IC₅₀) of about 40 micro-g/ml to about 80 micro-g/ml for inhibition of human acetylcholinesterase activity.

16. The method of claim 15, wherein the inhibition of human acetylcholinesterase is at least 5 times greater than the inhibition shown by urolithin A or urolithin B, separately.

17. The method of claim 16, wherein the therapeutically effective amount is in a daily dose from about 100 mg to about 1000 mg in a human.

18. The method of claim 16, wherein the therapeutically effective amount is in a daily dose from about 1.5 mg/kg to about 10.0 mg/kg in a human.
